(19) United States
(12) Patent Application Publication
(10) Pub. No.: US 2023/0252635 A1
(43) Pub. Date: Aug. 10, 2023

(54) RADIOGRAPHIC IMAGING APPARATUS, DECISION SUPPORT METHOD, AND RECORDING MEDIUM

(71) Applicant: KONICA MINOLTA, INC., Tokyo (JP)

(72) Inventors: Satoshi HASEGAWA, Tokyo (JP); Naoki HAYASHI, Tokyo (JP); Mao EGUCHI, Tokyo (JP); Keisuke KOEDA, Tokyo (JP)

(73) Assignee: KONICA MINOLTA, INC., Tokyo (JP)

(21) Appl. No.: 18/100,014

(22) Filed: Jan. 23, 2023

(30) Foreign Application Priority Data
Feb. 10, 2022 (JP) 2022-019208
Feb. 10, 2022 (JP) 2022-019212
Feb. 10, 2022 (JP) 2022-019216

(51) Int. Cl. G06T 7/00; A61B 6/00

(52) U.S. Cl. CPC G06T 7/0016; A61B 6/54; G06T 2207/10124; A61B 6/4405

(57) ABSTRACT

A radiographic imaging apparatus includes a hardware processor that acquires rounds imaging information related to radiographic imaging of an imaging subject when making rounds, and outputs, based on the acquired rounds imaging information, decision support information supporting a decision regarding whether radiograph data generated by the radiographic imaging satisfies a prescribed image quality.

### TECHNICAL FIELD

The present invention relates to a radiographic imaging apparatus, a decision support method, and a recording medium.

### DESCRIPTION OF THE RELATED ART

Conventionally, it is known that when a user such as a radiographer makes rounds of patients in a ward of a medical facility such as a hospital, rounds imaging is performed using a mobile radiographic imaging apparatus (rounds car) that takes radiographic (X-ray) images of the patients using a flat-panel detector (FPD) at the rounds location.

Compared to general radiography using a radiographic imaging apparatus permanently installed inside a general radiographic imaging room of a medical institution, rounds imaging is often performed on critically ill patients (immobile patients), and in many cases it is difficult to reproduce the previous imaging. For example, due to the circumstances of the bed and fixtures, the placement of the rounds car and the like fluctuates, and it is not possible to perform imaging with a fixed arrangement and sense of distance of the tube/FPD like in general radiography. In many cases, the user makes rounds alone, and must make a decision alone about whether to perform imaging. Moreover, there is a significant burden in the case of deciding that later image retake is necessary, as the rounds location may be in a different location, such as a ward or an operating room. Furthermore, since the communication network may be fragile or a wireless environment may be used for imaging at the rounds location, it may not be possible to acquire external information such as from a hospital information system (HIS)/radiology information system (RIS)/electronic medical records.

Accordingly, an X-ray imaging apparatus is known in which captured radiograph data is subjected to a gradation process using multiple different windows (center value and width of image density) to make it easier for an inexperienced radiographer to notice that adjusting the density of the captured image can eliminate the need for image retake due to imaging error (see JP 2010-172558A).

### SUMMARY OF THE INVENTION

In the X-ray imaging apparatus in JP 2010-172558A, a single decision criterion is proposed for inexperienced radiographers, in which captured images with different gradations are displayed as decision information for deciding whether imaging error exists, and a decision is made by applying specific processing to captured images.

However, in rounds imaging, the rounds location and the state of each patient are different every time, making it difficult to maintain an imaging environment similar to the previous imaging, and a change of gradation alone does not achieve reproducibility in many cases. For example, the above obviously pertains to patients receiving emergency care and undergoing an operation, but also to patients in a general ward who are unable to come to a general radiography room (are immobile), and the availability range (operating range) for radiographic imaging varies every time due to changes in the medical condition from day to day. Consequently, it may not be possible to make a decision regarding a radiographic imaging result according to a single criterion, and the user may need to judge criteria (evaluation criteria) in detail for each patient.

On the other hand, at the rounds location, unlike general radiography, there is no opportunity to consult with others, such as by consulting with other nearby radiographers or clinicians or referring to electronic medical records or a picture archiving and communication system (PACS). For this reason, to an inexperienced radiographer, the imaging error decision information for general radiography may be insufficient by itself, and as a result, the radiographer may have to perform the extra labor of revisiting a rounds location for an image retake after returning from rounds, or the radiographer may be unable to provide a radiograph of the quality sought by the clinician.

An objective of the present invention is to ensure the image quality level of radiograph data after rounds imaging has been performed (for example, to enable appropriate decision-making regarding whether image retake due to imaging error is necessary).

To achieve at least one of the abovementioned objects, according to an aspect of the present invention, a radiographic imaging apparatus reflecting one aspect of the present invention is provided with a hardware processor that acquires rounds imaging information related to radiographic imaging of an imaging subject when making rounds, and outputs, based on the acquired rounds imaging information, decision support information supporting a decision regarding whether radiograph data generated by the radiographic imaging satisfies a prescribed image quality.

According to an aspect of the present invention, a decision support method includes acquiring rounds imaging information related to radiographic imaging of an imaging subject when making rounds, and outputting, based on the acquired rounds imaging information, decision support information supporting a decision regarding whether radiograph data generated by the radiographic imaging satisfies a prescribed image quality.

According to an aspect of the present invention, a computer readable recording medium is provided, the medium storing a program causing a computer to function as a controller that acquires rounds imaging information related to radiographic imaging of an imaging subject when making rounds, and outputs, based on the acquired rounds imaging information, decision support information supporting a decision regarding whether radiograph data generated by the radiographic imaging satisfies a prescribed image quality.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic configuration diagram illustrating a radiographic imaging system according to an embodiment of the present invention;

FIG. 2 is a block diagram illustrating a configuration of an FPD;

FIG. 3 is a block diagram illustrating a configuration of a rounds car;

FIG. 4 is a diagram illustrating the structure of an imaging purpose table;

FIG. 5A is a block diagram illustrating the flow of data for a rounds car before radiographic imaging, and

FIG. 5B is a block diagram illustrating the flow of data for a rounds car after radiographic imaging;

FIG. 6 is a flowchart illustrating a first quality information acquisition process;

FIG. 7 is a flowchart illustrating a navigation process;

FIG. 8 is a diagram illustrating an imaging support screen;

FIG. 9 is a diagram illustrating a subject and a rounds car displaying a live preview image;

FIG. 10 is a flowchart illustrating a decision support process;

FIG. 11 is a diagram illustrating a decision support screen;

FIG. 12 is a diagram illustrating a decision support screen; and

FIG. 13 is a flowchart illustrating a second quality information acquisition process.

### DETAILED DESCRIPTION

An embodiment according to the present invention will be described in detail and with reference to the attached drawings. Note that the present invention is not limited to the illustrated examples.

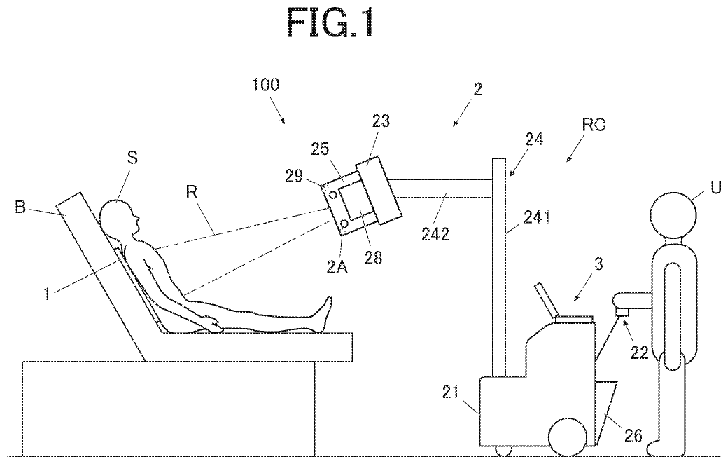

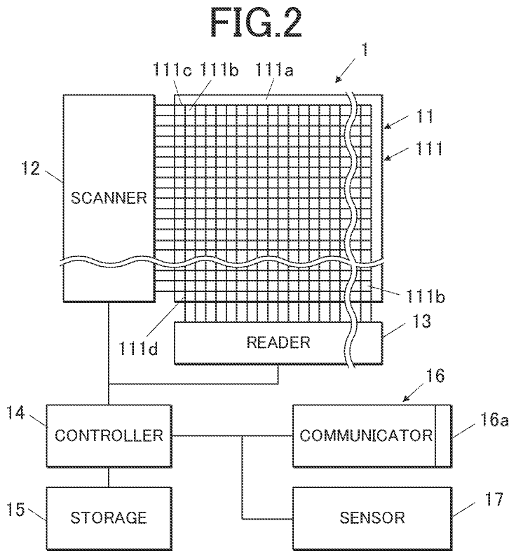

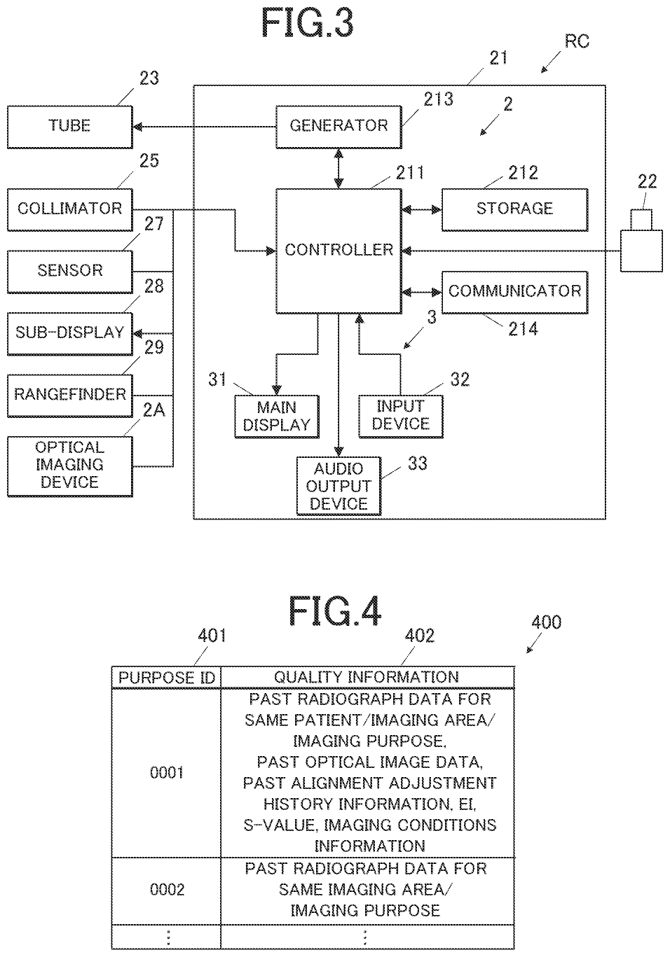

First, FIGS. 1 to 3 will be referenced to describe an apparatus configuration of the present embodiment. FIG. 1 is a schematic configuration diagram illustrating a radiographic imaging system 100. FIG. 2 is a block diagram illustrating a configuration of an FPD 1. FIG. 3 is a block diagram illustrating a configuration of a rounds car RC.

As illustrated in FIG. 1, the radiographic imaging system 100 that serves as a radiographic imaging apparatus of the present embodiment is provided in a medical facility such as a hospital. When a user U, such as a radiographer of the medical facility, makes rounds of patients to be visited, the radiographer takes the radiographic imaging system 100 to a rounds location where the patient is present, such as a ward or an operating room, and performs radiographic imaging of the patient. Herein, an example is described in which the rounds location is a ward, a bed B is installed in the ward, and the radiographic imaging is performed in a state with the subject S, that is, the patient who is the imaging subject, lying in a recumbent pose on the bed B of which the back has been raised.

The radiographic imaging system 100 is provided with an FPD I and a rounds car RC. The rounds car RC includes a radiation generator 2 and a console 3. The FPD 1 and the rounds car RC can communicate with each other through wireless communication, for example. Also, in a standby location (storage location) prior to making rounds, the rounds car RC is assumed to be directly connectible to a communication network (a local area network (LAN) or the like) internal to the medical facility, whereas at the rounds location, it is assumed that the rounds car RC is unable to connect to the communication network or that communication with the communication network is fragile.

In addition, the rounds car RC can communicate, through the communication network, with external apparatuses such as an HIS, an RIS 60, and an external apparatus 70 (FIGS. 5A, 5B). The communication network is assumed to be wireless, but may also be wired.

The FPD 1 is an apparatus that generates radiograph data corresponding to radiation R emitted from the radiation generator 2, and is panel-shaped and portable. Accordingly, the FPD 1 obviously can be used by being loaded onto an imaging platform, but may also be used by placing the FPD 1 horizontally between the bed B and the subject S lying supine on the bed B, or as illustrated in FIG. 1, may also be used by propping up the FPD 1 between a backrest and the subject S in a seated pose on the bed B with the back raised or in a wheelchair.

Note that the radiation incident surface (the surface facing the subject S) of the FPD 1 loaded onto an imaging platform is in a parallel or orthogonal state relative to the horizontal plane, but in imaging not involving an imaging platform (imaging on the bed B or in a wheelchair), the radiation incident surface may not necessarily be in a parallel or orthogonal state (may be inclined) relative to the horizontal plane. Also, in the state in which the FPD 1 is interposed between the subject S and a soft fixture such as the bed B, the FPD 1 may move in association with the movement of the subject S.

The radiation generator 2 is provided with a generator main body 21, an irradiation instruction switch 22, a tube 23, a tube support 24, a collimator 25, and an FPD storage compartment 26. Additionally, the radiation generator 2 is movable on wheels provided on a housing of the generator main body 21.

The irradiation instruction switch 22, when triggered by being operated (depressed) by the user U, outputs an operation signal to the generator main body 21. Note that although FIG. 1 illustrates an example of a state in which the irradiation instruction switch 22 is connected to the generator main body 21 in a wired manner, the irradiation instruction switch 22 and the generator main body 21 may also be connected in a wireless manner.

The tube 23, when triggered by the irradiation instruction switch 22 being operated, generates a dose of radiation R (such as X-rays) according to preset imaging conditions in a manner corresponding to the imaging conditions, and emits the radiation from an irradiation port.

The tube support 24 is an arm supporting the tube 23. The tube support 24 includes a support 241 extending from the generator main body 21 to an upper end and a support 242 extending forward from the upper part of the support 241. The leading end of the support 242 supports the tube 23. Additionally, through the inclusion of an articulating mechanism not illustrated in the tube support 24, the tube 23 can be moved in an X-axis direction (the front-back direction of the radiation generator 2 (left-right direction in FIG. 1)), a Y-axis direction (the width direction of the radiation generator 2 (the direction orthogonal to the page in FIG. 1)) orthogonal to the X axis, and a Z-axis direction (vertical direction (up-down direction in FIG. 1)) orthogonal to the X and Y axes. Moreover, through an articulating mechanism not illustrated, the tube support 24 can change the facing of the irradiation port of the radiation R by rotating the tube 23 about rotation axes parallel to the X, Y, and Z axes.

The collimator 25 is mounted to the irradiation port of the tube 23 and narrows the radiation R so that the irradiation field of the radiation R emitted from the irradiation port assumes a preset rectangular shape. The collimator 25 is also provided with a lamp button not illustrated. The operation of the lamp button by the user triggers the irradiation of visible light in the area that serves as the irradiation field of the radiation R.

The FPD storage compartment 26 is for storing the FPD 1 when not in use, and is provided on a lateral surface of the generator main body 21. Moreover, the FPD storage compartment 26 can accommodate the storage of multiple FPDs 1. A connector not illustrated is provided inside the FPD storage compartment 26, and may be configured such that if the FPD 1 is stored, the connector is connected to a connector 16a (FIG. 2) of the FPD 1.

The console 3 is configured as a personal computer (PC) or a mobile terminal, or as a dedicated device, and is mounted on top of the radiation generator 2. The console 3 can set imaging conditions (such as the tube voltage, tube current and irradiation time or current-time product (mAs value), imaging area, and imaging direction) in at least one of the FPD 1 and the radiation generator 2 on the basis of an imaging order acquired from an external apparatus (such as the RIS 60 (FIG. 5A)) or an operation performed on an input device 32 by the user U. The imaging order is information pertaining to radiographic imaging that a clinician requests from the user, and includes information such as a designated date and time of radiographic imaging, subject information (such as a patient ID described later) about the subject to be imaged, the imaging area (such as an imaging area ID described later), a purpose ID described later, and imaging details. Also, the console 3 can acquire radiograph data generated by the FPD 1 and save the data internally or transmit the data to another external apparatus (such as the PACS).

Radiographic imaging (seated imaging) using the radiographic imaging system 100 (rounds car RC) configured in this way is performed as follows. First, the user U disposes the radiographic imaging system 100 beside the vicinity (bed B) of the subject S. Thereafter, the user U asks the subject S to assume a seated pose. If the subject S is sitting in a fixture that allows for angle adjustment (such as the bed B that can be partially raised), the user U appropriately adjusts the angle of the backrest of the bed B. Thereafter, the rough position and direction of the tube 23 are adjusted such that the irradiation port of the tube 23 points in the direction of the imaging area of the subject S. The FPD 1 is then retrieved from the FPD storage compartment 26 and placed between the back of the subject S and the backrest. The direction and irradiation field of the tube 23 are then finely adjusted so that the irradiation axis of the radiation R is orthogonal to the radiation incident surface of the FPD 1. Thereafter, radiographic imaging is performed (the imaging area of the subject S is irradiated with the radiation R, causing the FPD 1 to generate radiograph data of a still image or a dynamic image of the area to be diagnosed).

Note that the generator main body 21 and the console 3 are assumed to have an integrated configuration (or stored in a single housing), but may also be separate. Also, the radiation generator 2 may be movable by means other than wheels. For example, the radiation generator 2 may be lightweight enough to be carried by a person or mountable on a commercially available trolley or the like, or the undersurface may be smooth enough to slide across the floor. Moreover, in the radiographic imaging system 100, one of the FPD 1 and the radiation generator 2 may also be a stationary installation in a room or the like of the medical facility (while the other is freely movable).

Next, FIG. 2 will be referenced to describe an internal configuration of the FPD 1. As illustrated in FIG. 2, the FPD 1 is provided with a radiation detector 11, a scanner 12, a reader 13, a controller 14, storage 15, a communicator 16, and a sensor 17. The components of the FPD 1 are communicably connected.

The radiation detector 11 is provided with a scintillator (not illustrated) and a photoelectric conversion panel 111. The scintillator is formed into a flat plate of columnar CsI crystal, for example. Additionally, in response to receiving radiation, the scintillator emits electromagnetic waves (visible light, for example) having a longer wavelength than the radiation at an intensity corresponding to the dose (mAs) of the received radiation. Also, the scintillator is disposed so as to extend parallel to the radiation incident surface of the housing of the radiation detector 11.

The photoelectric conversion panel 111 is disposed to extend parallel to the scintillator on the opposite side from the surface of the scintillator that faces the radiation incident surface. The photoelectric conversion panel 111 includes a board 111a and a plurality of charge storage components 111b. The plurality of charge storage components 111b are arrayed on the surface of the board facing the scintillator, in a two-dimensional shape (for example, a matrix of rows and columns) corresponding to each pixel of a radiograph. The charge storage components 111b each include a semiconductor element that generates a quantity of charge corresponding to the intensity of the electromagnetic waves produced by the scintillator, and a switch element provided between each semiconductor element and a wiring line connected to the reader 13. A bias voltage is applied to each semiconductor element from a power supply circuit not illustrated. Additionally, by toggling the on/off state of the switch element, each charge storage component stores and releases a charge to be read out as a signal value according to received radiation.

The scanner 12 toggles the on/off state of each switch element by applying an ON voltage or an OFF voltage to each scan line 111c of the radiation detector 11.

The reader 13 reads out the quantities of charge flowing in from the charge storage components 111b through each signal line 111d of the radiation detector 11 as signal values. Note that the reader 13 may also perform binning when reading out signal values.

The controller 14 includes a central processing unit (CPU) not illustrated and random access memory (RAM) (not illustrated). The CPU reads out and loads various processing programs stored in the storage 15 into the RAM and executes various processes in cooperation with the loaded processing programs to thereby centrally control operations by each component of the FPD 1. Also, the controller 14 generates radiograph data on the basis of a plurality of signal values read out by the reader 13.

The storage 15 is configured as a semiconductor memory, a hard disk drive (HDD), and/or the like, and stores various programs to be executed by the controller 14, parameters required for the execution of the programs, and various data in files. Note that the storage 15 may also store the image data of radiographs.

The communicator 16 is configured as a communication module for wireless communication or the like. Additionally, the communicator 16 transmits and receives various signals and various data with respect to an external apparatus such as the rounds car RC connected by wireless communication.

The sensor 17 is a detector of information necessary for calculating the angle of opposition between the FPD 1 and the tube 23. The sensor 17 is a 3-axis accelerometer. A 3-axis accelerometer detects the acceleration acting in each of three axial (x-axis, y-axis, and z-axis) directions, and outputs acceleration information regarding the detected accelerations in the three axial directions to the controller 14. In a stationary state, only gravitational acceleration acts upon the 3-axis accelerometer. Accordingly, in a stationary state, the 3-axis accelerometer detects each of the three axial-direction components of gravitational acceleration.

Note that the sensor 17 may also be a 6-axis sensor or a 9-axis sensor. A 6-axis sensor is the above 3-axis accelerometer with the addition of a function for detecting the angular velocity (gyro) for each of the three axes. Also, a 9-axis sensor is the above 6-axis sensor with the addition of a function for detecting the direction (north, south, east, west) of each of the three axes.

The controller 14, when triggered by the fulfillment of a predetermined condition, for example, causes the sensor 17 to repeatedly detect acceleration information regarding the gravitational acceleration in the three axial directions. The predetermined condition encompasses, for example, the detector 1 being powered on, a predetermined control signal being received from another apparatus (such as the rounds car RC), or a predetermined operation being performed on an input device (not illustrated) of the FPD 1. Every time the sensor 17 detects acceleration information regarding the gravitational acceleration, the controller 14 transmits the detected acceleration information about the gravitational acceleration to the rounds car RC through the communicator 16.

As an example of operations by the controller 14, the controller 14 executes control causing the scanner 12 to store/release electric charges in the radiation detector 11 in synchronization with the timing at which the radiation R is emitted from the radiation generator 2. The controller 14 also executes control causing the reader 13 to read out signal values based on the electric charges released by the radiation detector 11. The controller 14 also generates the radiograph data of a still image or a dynamic image according to the dose distribution of the emitted radiation R, on the basis of the signal values read out by the reader 13. In the case of generating the radiograph data of a still image, the controller 14 generates radiograph data only once in response to a single press of the irradiation instruction switch 22. In the case of generating the radiograph data of a dynamic image, the controller 14 generates the radiograph data of a frame constituting a dynamic image multiple times per a given unit of time (for example, 15 times a second) in response to a single press of the irradiation instruction switch 22. Additionally, the controller 14 transmits the generated radiograph data to an external apparatus (such as the rounds car RC) through the communicator 16.

Note that the FPD 1 is not limited to being of the indirect conversion type in which radiation is converted into an electrical signal via a scintillator as above, and may also be of the direct conversion type in which radiation is converted into an electrical signal directly by semiconductor elements.

Next, FIG. 3 will be referenced to describe the internal configuration of the radiation generator 2 and the console 3 of the rounds car RC. As illustrated in FIG. 3, besides being provided with the generator main body 21, irradiation instruction switch 22, tube 23, tube support 24, collimator 25, and FPD storage compartment 26, the radiation generator 2 is provided with a sensor 27, a sub-display 28, a rangefinder 29, and an optical imaging device 2A. Additionally, the generator main body 21 includes a controller 211 (hardware processor), storage 212, a generator 213, and a communicator 214. The components of the radiation generator 2 are communicably connected, except for the tube 23.

The sensor 27 is provided in the tube 23 and is a 3-axis accelerometer similar to the sensor 17. Note that the sensor 27 may also be a 6-axis sensor or a 9-axis sensor. Moreover, the sensor forming the sensor 27 may also be a different type from the sensor forming the sensor 17. The controller 211 calculates the angle of opposition between the FPD 1 (the radiation incident surface thereof) and the tube 23 (the surface perpendicular to the radiation irradiation direction thereof) from acceleration information about the gravitational acceleration in three axial directions received from the stationary FPD 1 through the communicator 214 and acceleration information about the gravitational acceleration in three axial directions detected by the sensor 27 in the stationary tube 23.

The sub-display 28 is configured as a display such as a liquid crystal display (LCD) panel or an organic light-emitting diode (OLED) display panel, and is provided near the tube 23, for example. The sub-display 28 displays various images and other display information in accordance with display information inputted from the controller 211. The sub-display 28 and the optical imaging device 2A are assumed to be provided in the housing of the collimator 25. Note that the sub-display 28 and the optical imaging device 2A may also be configured to be provided in the housing of the tube 23 or in the tube support 24. Moreover, the content to be displayed by the sub-display 28 is differentiated from the content to be displayed by the main display 31.

The rangefinder 29 is an instrument that measures the source-to-image distance (SID) and outputs the measured SID to the controller 211. The SID is the distance between the focal point F of the radiation R and the imaging surface of the FPD 1 (the surface of the radiation detector 11 on which the charge storage components 111b are provided). Note that the rangefinder 29 may also be configured to measure the source-to-skin distance (SSD). The SSD is the distance between the focal point F of the radiation R and the body surface of the subject S, and is substantially equal to the difference between the SID and the body thickness of the subject S. The rangefinder 29 is provided in the collimator 25.

The rangefinder 29 may be configured to include light-emitting means for emitting laser light, detecting means for detecting reflected laser light, and calculating means for calculating the distance from the light-emitting means to the reflection point on the basis of the time from when the laser light is emitted until the reflected laser light is detected. The rangefinder 29 may also be configured to include calculating means for calculating the SID on the basis of an optical image of the FPD I generated by the optical imaging device 2A optically imaging the FPD 1 in the irradiation direction of radiation and size information about the FPD 1. The rangefinder 29 may also include a combination of the above. Also, since laser light is reflected by the body surface of the subject S, the distance measured by the rangefinder 29 using laser light is the SSD in many cases. In such cases, the value obtained by adding the body thickness of the subject S to the measured SSD may be taken to be the SID. The body thickness may be a prescribed reference value, a numerical value inputted by the user U, or a value calculated automatically from information about the subject S.

The controller 211 calculates alignment information, including 3-axis acceleration information (from which is calculated inclination information (pose) about the radiation incident surface of the FPD 1 relative to the horizontal plane) about the FPD 1 according to the sensor 17, 3-axis acceleration information (from which is calculated inclination information (pose) about the surface of the tube 23 (collimator 25) perpendicular to the radiation irradiation direction relative to the horizontal plane) about the tube 23 according to the sensor 27, and the distance between the FPD 1 and the tube 23 according to the rangefinder 29. The inclination information about the FPD 1 may also be expressed as a difference value from the inclination information about the tube 23 (collimator 25). The user can understand the arrangement of the FPD 1 and the tube 23 for the current radiographic imaging from alignment information about radiographic imaging performed in the past, and adjust the arrangement of the FPD 1 and the tube 23 (collimator 25) accordingly. Note that the alignment information may also be standalone alignment with only inclination information about the tube 23.

The optical imaging device 2A includes optics such as a lens, and an image sensor such as a charge-coupled device (CCD) sensor or a complementary metal-oxide-semiconductor (CMOS) sensor. The optical imaging device 2A, under control by the controller 211, optically images the subject S as the imaging subject with visible light to generate and output optical image data to the controller 211 and the like. For example, the optical imaging device 2A optically images the subject S to generate the optical image data of a still image or a dynamic image (such as a live preview image).

The controller 211 includes a CPU, RAM, and/or the like. In addition, the CPU reads out and loads various programs stored in the storage 212 into the RAM and executes various processes in cooperation with the loaded programs to thereby control the components of the radiation generator 2 and the console 3.

The storage 212 is configured as a non-volatile memory, HDD, and/or the like, and stores various programs to be executed by the controller 211, parameters required for the execution of the programs, and various data such as files. In particular, a first quality information acquisition program for executing a first quality information acquisition process described later, a navigation program for executing a navigation process described later, a decision support program for executing a decision support process described later, and an imaging purpose table 400 described later are assumed to be stored in the storage 212.

The generator 213, when triggered by receiving an imaging instruction signal from the controller 211, applies to the tube 23 a voltage according to preset imaging conditions and energizes the tube 23 with a current according to the imaging conditions.

The communicator 214 is configured as a communication module or the like. The communicator 214 can transmit and receive various signals and various data with respect to an external apparatus such as the wirelessly connected FPD 1 or the RIS 60 over a communication network.

The console 3 is provided with a controller, storage, a communicator, a main display 31, an input device 32, and an audio output device 33. The controller, storage, and communicator of the console 3 double as the controller 211, storage 212, and communicator 214, respectively, of the radiation generator 2. Note that the console 3 may also be configured to include a dedicated controller, storage, and communicator.

The main display 31 is configured as an LCD, OLED, or other display panel. The main display 31 displays various information in accordance with display information inputted from the controller 211.

The input device 32 is configured as a keyboard with various keys, a pointing device used to input position information, or a touch panel integrated with the display screen of the main display 31, for example, accepts operation input from the user U, and outputs corresponding operation information to the controller 211.

The audio output device 33 is configured as an amplifier, a speaker, and/or the like, and outputs audio according to audio information inputted from the controller 211. For example, the audio output device 33 outputs synthesized speech of a message as imaging support information, that is, information for supporting the user U with radiographic imaging. The imaging support information is information based on quality information, and is information that supports (assists with) radiographic imaging of a subject to be imaged to improve the image quality of the radiograph data. The quality information is information for evaluating the image quality of radiograph data according to the imaging purpose of the radiographic imaging.

Next, FIG. 4 will be referenced to describe information stored in the rounds car RC. FIG. 4 is a diagram illustrating the structure of an imaging purpose table 400.

The imaging purpose table 400 is assumed to be stored in the storage 212 of the rounds car RC. The imaging purpose table 400 is a table defining quality information pertaining to the image quality according to imaging purpose that the rounds car RC acquires by radiographic imaging when making rounds. The imaging purpose refers to the type of consultation which is the purpose of using radiograph data of a subject imaged when making rounds, and may be “follow-up observation”, “initial consultation”, “disease identification”, “checking the location of intubation object such as a tube”, “status checkup on patient transported by ambulance”. Also, in one configuration, the imaging purpose may be different depending on the medical department and the type of disease. Moreover, in cases in which wards are separated by medical department when making rounds of the wards, it is also possible to narrow down the imaging purpose to some extent by identifying the ward instead of the medical department.

Image quality refers to the quality at which the visibility of lesions, medical equipment such as catheters, and the like in the captured radiograph data is sufficient for the examination and diagnostic purposes by a clinician, and/or the quality that fulfills the requirements enabling the execution of image analysis processing using the radiograph data (including dynamic images). The requirements pertain to, for example, signal values, granularity, contrast, the positioning of the area to be analyzed, and the presence or absence of streaks and artifacts. For example, in the case of radiograph data of a dynamic image, the image quality encompasses whether a certain number of frames fulfilling the requirements have been obtained, whether the minimum imaging duration (frames) necessary for image analysis has been obtained, whether subject movement (such as respiration) continues or is stopped for a certain period of time, whether bare portions exist, and whether the frame rate is suitable. Bare portions refer to portions of the radiograph data where the imaging subject (the imaging area of the subject) does not exist (portions where there is only air). Being absent of any obstacles, bare portions are exposed to a high dose, and thus if a pixel value comparison is performed together with the range of the imaging subject, correct values such as the mean and the maximum will not be obtained. Therefore, when performing analysis, calculations are made by excluding bare portions and artificial objects, for example. Note that even for the same imaging area (imaging order), the observation area and the radiograph data to be compared (such as past radiograph data from the same patient or radiograph data from another patient) may be different depending on the imaging purpose, and the decision metric regarding imaging error in the radiograph data after imaging is also different. The quality information is not information for setting imaging conditions like in JP 2010-172558A, but rather is information used to evaluate the image quality of the captured radiograph data.

As illustrated in FIG. 4, the imaging purpose table 400 includes items (columns) of a purpose ID 401 and quality information 402. The purpose ID 401 is identification information pertaining to the imaging purpose of radiographic imaging. The quality information 402 is the content of the quality information that the rounds car RC acquires from an external apparatus (the RIS 60 (FIG. 5A)) with respect to the imaging purpose of the purpose ID 401 (and the imaging area). In the example of the quality information 402 illustrated in FIG. 4, “patient” is the subject. “Imaging area” is the area of the subject to be imaged, and is thoracic recumbent AP (radiographic 20 imaging in which the subject is irradiated with radiation from the anterior (ventral) side to the posterior (dorsal) side). “Alignment adjustment history” is history information regarding the adjustment of alignment information pertaining to the tube 23 and the FPD 1. “Exposure index (EI)” is an index indicating the incident dose on the FPD 1. “S-value” is a sensitivity corresponding to the radiation dose during radiographic imaging.

For example, in the imaging purpose table 400, the purpose ID 401 is taken to be “0001” in the case in which the imaging area is thoracic recumbent AP and the imaging purpose is “follow-up observation”. Also, the purpose ID 401 is taken to be “0002” in the case in which the imaging area is thoracic recumbent AP and the imaging purpose is “initial consultation”. Note that the purpose ID 401 (quality information) may also be configured to be associated with at least one of an imaging area (imaging area ID), a subject (patient ID), a user (user ID of radiographer), and a clinician (clinician ID). In particular, the quality information for the imaging support information is taken to be information related to an imaging method for radiographic imaging corresponding to at least one of an imaging area (imaging area ID), a subject (patient ID), a user (user ID of radiographer), and a clinician (clinician ID). In FIG. 4, the quality information 402 for a purpose ID 401 is illustrated as having multiple items as an example, but IDs may also be assigned to single pieces of quality information, and the IDs may be grouped together by the purpose ID. Also, the units of each item, a settable range (or threshold value), and the like may also be defined in a form relative to the quality information 402. For example, for “past” data, there is a setting that specifies how many generations back are subject to examination.

An example of imaging purposes, quality information, and decision support information in the present embodiment is illustrated in the following Table 1. Decision support information refers to information which is based on the quality information, which is support (assistance) information for a user to decide whether radiograph data captured by radiographic imaging satisfies a prescribed image quality (whether image retake due to imaging error is necessary), and which is information for improving the image quality of radiograph data.

TABLE 1
Table I

| Imaging purpose | Quality information | Decision support information |
|---|---|---|
| Progress of improvement | Image data to be compared | Whether target location is |
| due to treatment/therapy | (past image/clinical image), | included in image, whether image |
| | ROI information, | quality allows for determination |
| | case information to be checked | of inflammatory response |
| Sideeffects of | Image data to be compared | Whether surroundings including |
| treatment/therapy | (past image/clinical image), | target area are included in image, |
| | ROI information, | whether image quality allows for |
| | case information to be checked | determination of side effects |
| | | (e.g., complications) |
| Checking tube position | Object information to be checked (tube) | Whether tube appears in image |
| Joint (knee joint, elbow joint, | ROI information, case information to be | Positioning |
| ankle joint) imaging | checked (direction/angle) | (e.g., in knee case, lateral/medial malleolus |
| | | overlap/left-right mistake/wrong area) |
| Limb (arm, leg) imaging | Comparative information | Left-right mistake, wrong area |
| | (normal image), ROI information, | |
| | case information to be checked | |
| | (area in imaging conditions) | |
| Torso (abdomen, spine, | ROI information, optimal dose setting | Saturation due to overdose, body |
| hip joint) imaging | information for each area, area | movement, wrong area |
| | information (body thickness) | |
| Thoracic imaging | Image data to be compared | Missing lung field, wrong area, |
| | (past image/clinical image), | lung field region shape, position, |
| | ROI information, case information to be | size, inclination |
| | checked, selected panel information | |
| | (size/direction) | |
| Disease identification | Image data to be compared | Radiograph in exhalation phase |
| (e.g., pneumothorax) | (normal image), case information to be | |
| | checked, other examination information | |
| | (spirometry) | |
| Emergency | Clinician request | Imaging over wide area |
| Follow-up observation | Image data to be compared | Information regarding time lapse |
| | (past image/clinical image), | of past images |
| | ROI information, case information to be | |
| | checked, grid information | |
| Imaging that includes metal | Information regarding presence of | Artifacts |
| | metal inside body, ROI information | |
| | (location containing metal) | |
| Imaging of thick-bodied | Patient information, | Image quality (e.g., EI, S-value) |
| individual | past irradiation information | |
| Surgery | Preoperative image data | |
| Clinician demand | Clinician demand information, | Degree of alignment and area of |
| | failed image data (image with error) | importance, alert level and |
| | | threshold value |

In Table I, the image data (image) is radiograph data (a radiograph), but may also be optical image data (an optical image).

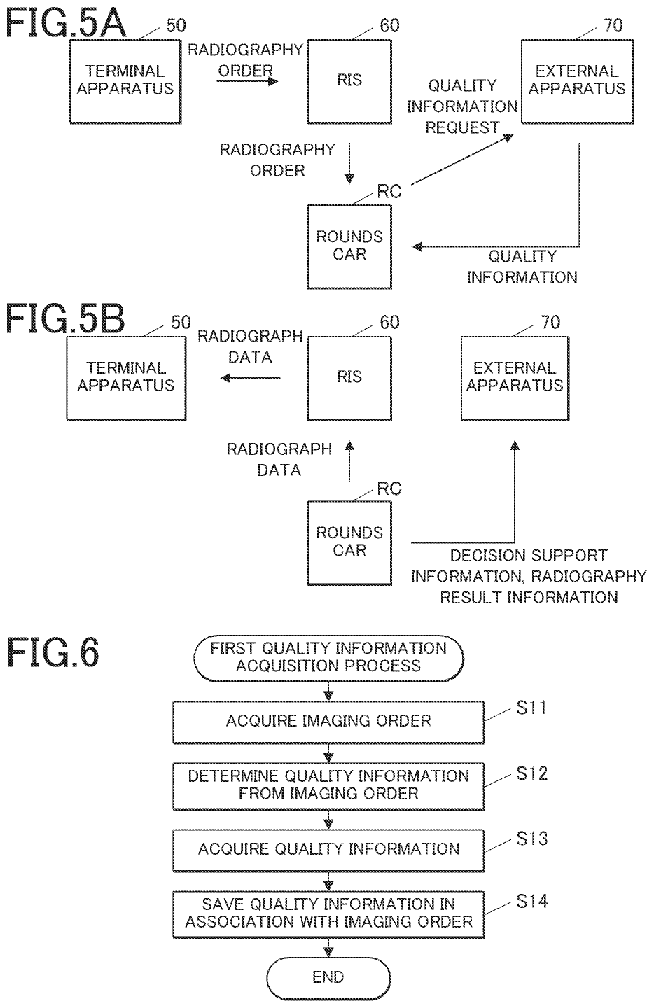

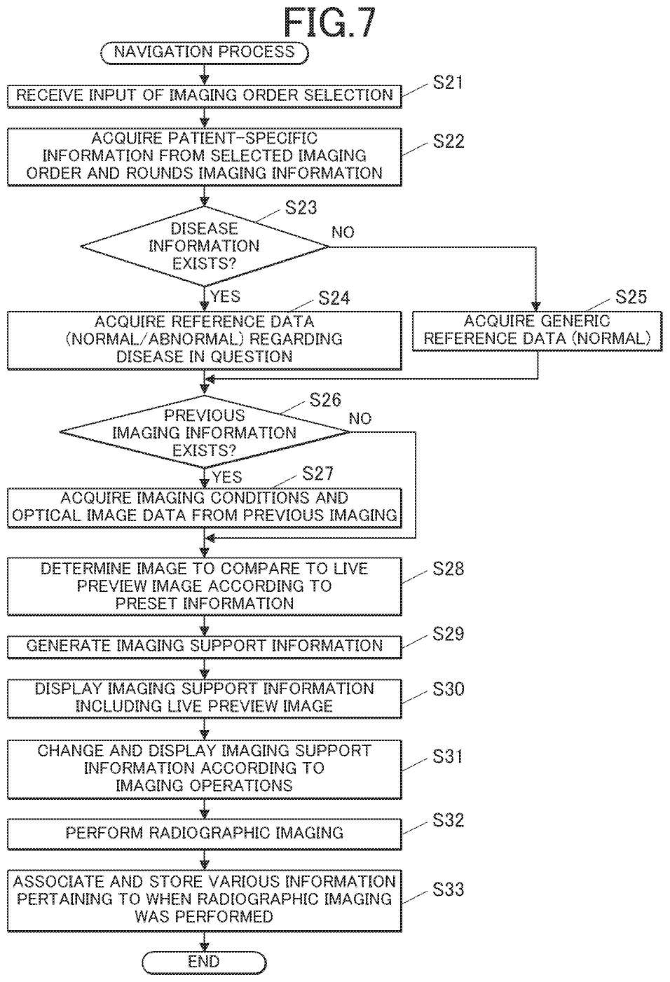

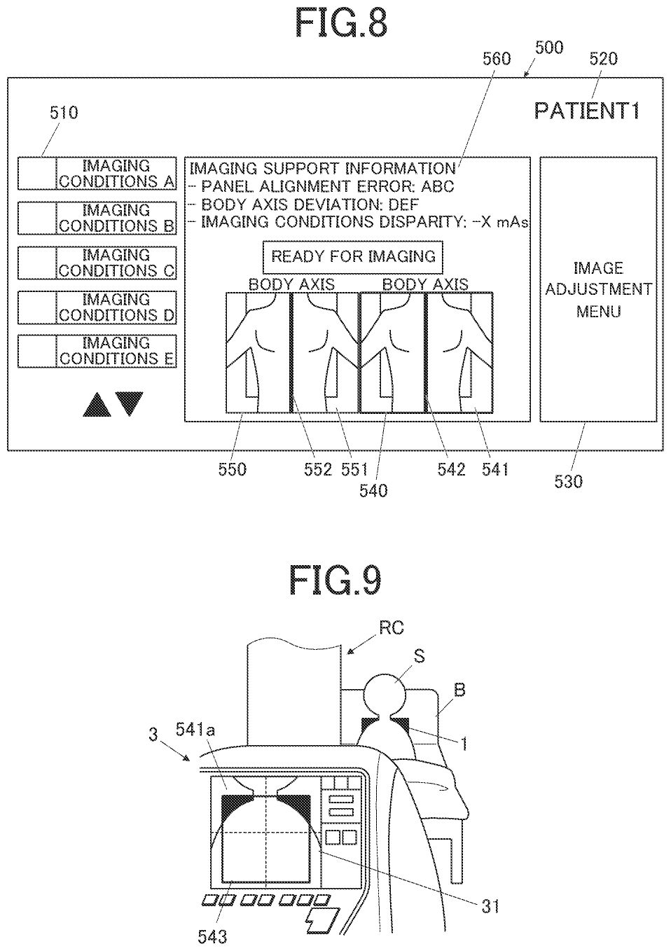

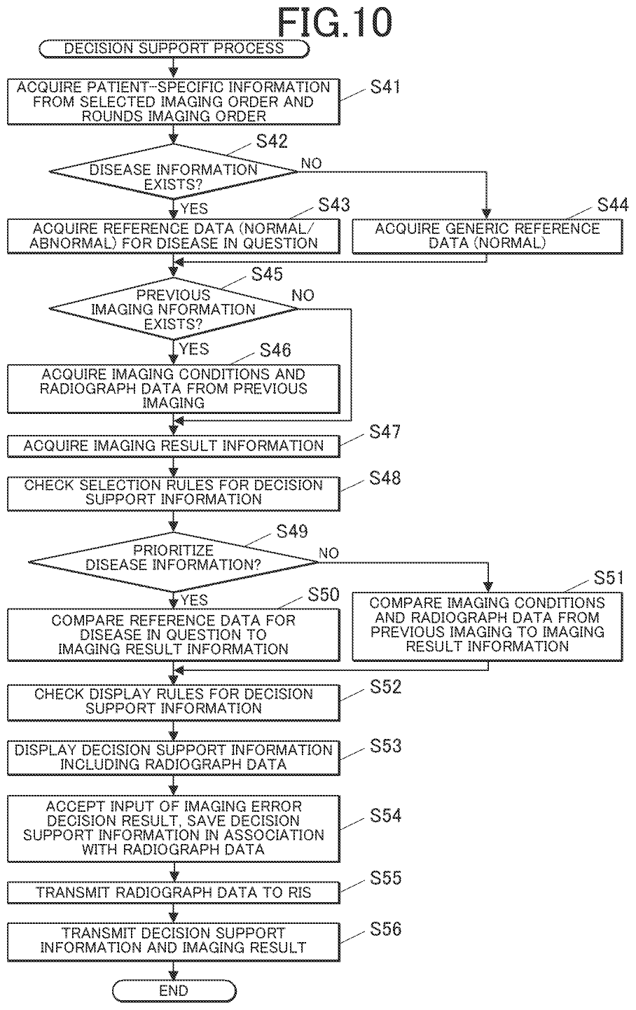

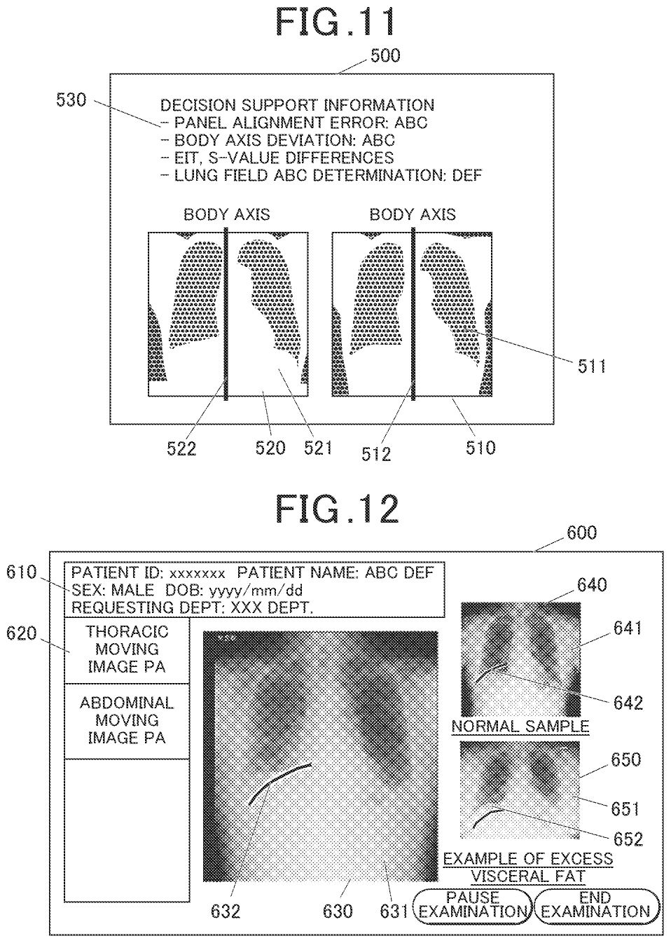

Next, FIGS. 5A to 12 will be referenced to describe operations by the radiographic imaging system 100. FIG. 5A is a block diagram illustrating the flow of data for the rounds car RC before radiographic imaging. FIG. 5B is a block diagram illustrating the flow of data for the rounds car RC after radiographic imaging. FIG. 6 is a flowchart illustrating a first quality information acquisition process. FIG. 7 is a flowchart illustrating a navigation process. FIG. 8 is a diagram illustrating an imaging support screen 500. FIG. 9 is a diagram illustrating a subject S and a rounds car RC displaying a live preview image 541a. FIG. 10 is a flowchart illustrating a decision support process. FIG. 11 is a diagram illustrating a decision support screen 500. FIG. 12 is a diagram illustrating a decision support screen 600.

As illustrated in FIG. 5A, in a medical facility, a terminal apparatus 50, an RIS 60, an external apparatus 70, and a rounds car RC in a standby location are communicably connected in advance through a relay apparatus (such as an access point connected to a communication network, another rounds car, or a console). The terminal apparatus 50 is a terminal apparatus such as a PC used by a clinician. The RIS 60 is a server of a radiology information system (RIS), but may also be a server of a hospital information system (HIS). The external apparatus 70 is a server that manages quality information, in which quality information is stored. The external apparatus 70 may also overlap with the RIS 60 or an HIS.

In the case of performing radiographic imaging on a subject at a rounds location, the terminal apparatus 50 receives operation input such as the date and time of the radiographic imaging, designation information designating at least one subject (patient) to be imaged, the imaging area, and the image purpose from a clinician, and issues an imaging order, including the imaging date and time, a patient ID corresponding to the patient designation information, an imaging area ID corresponding to the imaging area, and a purpose ID corresponding to the imaging purpose, to be transmitted to the RIS 60. The imaging order issued by the terminal apparatus 50 is an order for a single unit of imaging subject to imaging conditions and the like, and therefore serves as an imaging unit (image unit). The RIS 60 receives and stores the imaging order from the terminal apparatus 50 in internal storage, and issues an imaging order (RIS order), which is based on the received imaging order, to be transmitted to the rounds car RC. The imaging order (RIS order) issued by the RIS 60 is an order for a single unit of examination by radiographic imaging, and generally serves as an examination order. An examination order is an order for a single unit of examination, and contains a plurality of imaging orders (issued by the terminal apparatus 50).

A user such as a radiographer goes to the standby location where the FPD 1 and the rounds car RC are located. Thereafter, the controller 211 of the rounds car RC (console 3), when triggered by the start of receiving an imaging order from the RIS 60 through the communicator 214, for example, executes the first quality information acquisition process by following the first quality information acquisition program stored in the storage 212.

As illustrated in FIG. 6, first, the controller 211 finishes receiving and thereby acquires the imaging order from the RIS 60 through the communicator 214 (step S 11). Thereafter, the controller 211 references the imaging purpose table 400 stored in the storage 212 and determines the quality information to be acquired from the quality information 402 corresponding to the purpose ID 401 in the imaging order acquired in step SI I (step S12).

Next, the controller 211 transmits, to the external apparatus 70 through the communicator 214, a request for the quality information determined in step S12 and including the patient ID, the purpose ID, and the imaging area ID of the imaging order, and receives and acquires the quality information from the external apparatus 70 (step S13). In response to step S13, the external apparatus 70 receives the request for quality information from the rounds car RC, retrieves from internal storage the quality information corresponding to the patient ID, the purpose ID, and the imaging area ID included in the request for quality information, and transmits the retrieved quality information to the rounds car RC.

Thereafter, the controller 211 saves the quality information acquired in step S13 in association with the imaging order in the storage 212 (step S14), and ends the first quality information acquisition process.

Note that in the case of a communication environment in which the communication network is also usable at the rounds location or on the path between the standby location and the rounds location, the controller 211 of the rounds car RC may also be configured to execute the first quality information acquisition process at the rounds location or on the way from the standby location to the rounds location, rather than at the standby location.

After the execution of the first quality information acquisition process, the user takes the rounds car RC storing the FPD 1 from the standby location to the rounds location where the subject to be imaged is present, and initiates radiographic imaging of the subject. At this time, the controller 211 of the rounds car RC (console 3) at the rounds location, when triggered by the input of an instruction for executing the navigation process from the user through the input device 32, for example, executes the navigation process by following the navigation program stored in the storage 212.

As illustrated in FIG. 7, first, the controller 211 retrieves all imaging orders from the storage 212 and displays them on the main display 31, and accepts, through the input device 32, input from the user for selecting the imaging order for the subject (patient) to be imaged from among the displayed imaging orders (step S21).

Next, the controller 211 retrieves and acquires from the storage 212 the quality information corresponding to (the subject (patient) of) the imaging order selected and inputted in step S21, acquires rounds imaging information including the acquired quality information and various information pertaining to the current radiographic imaging, and acquires, from the selected imaging order and the acquired rounds imaging information, patient-specific information that serves as information specific to the subject to be imaged (step S22). The patient-specific information is assumed to include at least one of disease information indicating a disease of the subject (patient) and previous imaging information that serves as various information pertaining to when radiographic imaging was performed on the subject previously.

Next, the controller 211 determines whether disease information about the subject to be imaged exists among the patient-specific information acquired in step S22 (step S23). If disease information exists (step S23; YES), the controller 211 acquires, from the quality information (rounds imaging information) acquired in step S22, reference data (condition of disease is normal/abnormal) about other patients in the past corresponding to the disease information in step S23 (step S24). The reference data (normal/abnormal) in step S24 includes optical image data from when radiographic imaging (normal/abnormal) was performed on other patients having the disease in the past, imaging conditions such as alignment information for rounds imaging, patient information (physique/build) about the subject, and the like.

If disease information does not exist (step S23; NO), the controller 211 acquires, from the quality information (rounds imaging information) acquired in step S22, generic reference data (condition of disease is normal) about other, healthy patients in the past (step S25). The reference data (normal) in step S25 includes optical image data from when radiographic imaging (normal) was performed on other patients in the past, imaging conditions such as alignment information for rounds imaging, patient information (physique/build) about the subject, and the like.

Next, the controller 211 determines whether previous imaging information about the subject to be imaged exists among the patient-specific information acquired in step S22 (step S26). If previous imaging information exists (step S26; YES), the controller 211 acquires, from the previous imaging information in step S26, imaging conditions such as alignment information pertaining to when radiographic imaging was performed previously, optical image data, and the like (step S27).

Thereafter, the controller 211 determines, according to preset information, the optical image data to be compared to a live preview image (video) of the subject captured by the optical imaging device 2A from among the optical image data acquired in steps S24, S25, and S27 (step S28). The preset information is setting information that has been set in advance, and is for example selection setting information pertaining to which optical image data of a diseased patient, a healthy patient, or the subject to be imaged is to be prioritized for comparison. Note that in step S28, the controller 211 may also be configured to accept, from the user through the input device 32, selection input indicating which of the optical image data acquired in steps S24, S25, and S27 is to be compared to the live preview image. If previous imaging information does not exist (step S26; NO), the flow proceeds to step S28.

Next, the controller 211 generates, from the optical image data to be compared that was determined in step S28 and the rounds imaging information, imaging support information supporting radiographic imaging of the subject to be imaged (step S29). The rounds imaging information in step S29 is information pertaining to the current radiographic imaging, and includes information about the alignment (panel alignment) between the FPD 1 and the tube 23, and the dose of the tube 23, for example.

Thereafter, the controller 211 displays, on the main display 31, the imaging support information generated in step S29, including optical image data of a live preview image of the subject acquired through optical imaging by the optical imaging device 2A (step S30). In step S30, the imaging support screen (navigation screen) 500 illustrated in FIG. 8 is displayed, for example.

The imaging support screen 500 is an example of an imaging support screen for radiographic imaging of the chest as the imaging area of the subject. The imaging support screen 500 includes imaging conditions buttons 510, a patient information display area 520, an image adjustment menu area 530, a live preview image area 540, a comparative optical image area 550, an imaging support information area 560, and the like.

The imaging conditions buttons 510 are buttons provided according to the imaging order, and are buttons for setting the imaging conditions (radiation irradiation conditions and radiograph reading conditions) for each instance of radiographic imaging and applying the imaging conditions to the radiation generator 2 and the FPD 1. For each imaging conditions button 510, an imaging conditions name expressing the content of the imaging conditions is displayed. The patient information display area 520 is an area in which patient information (here, a patient name) about the patient (subject) to be imaged is displayed. The image adjustment menu area 530 is an area in which is displayed an image adjustment menu for an optical image (live preview image 541) displayed in the live preview image area 540.

The live preview image area 540 is a display area for the live preview image 541 that serves as an optical image of the imaging area (back) of the subject captured through optical imaging by the optical imaging device 2A. The live preview image 541 is an optical image of a live preview of the back of the subject to be imaged. A body axis assistance line 542 of the subject generated through image analysis of the live preview image 541 by the controller 211 is superimposed onto the live preview image 541. Note that, as illustrated in FIG. 9, the controller 211 may also be configured to display a live preview image 541a on the main display 31. The live preview image 541a is an image in which an FPD perimeter outline 543 generated by the controller 211 is drawn on the live preview image looking down on the subject S, for example. The FPD perimeter outline 543 is an outline of a position suitable for radiographic imaging that serves as a reference for the perimeter (outline) of the FPD 1 inserted behind the subject S. The user performs radiographic imaging by adjusting the position and angle of the optical imaging device 2A and the position of the rounds car RC while visually checking the live preview image 541a, so that the live preview image of the FPD 1 is within the FPD perimeter outline 543. Note that in another possible configuration, the live preview image 541 may be displayed on the imaging support screen 500 instead of the live preview image 541a.

The comparative optical image area 550 is a display area for a comparative optical image 551 that serves as an optical image of quality information optically captured when radiographic imaging was performed on the imaging area (back) of another patient in the past. A body axis assistance line 552 of the patient as the imaging subject is superimposed onto the comparative optical image 551. Also, the live preview image area 540 and the comparative optical image area 550 are arranged side-by-side horizontally to allow for easy comparison by the user. Note that if a user interface (UI) not illustrated is operated, or if a display rule is set in advance, the transparency of either the live preview image 541 or the comparative optical image 551 may be adjusted such that the two images can be superimposed and displayed as a composite image.

The imaging support information area 560 is a display area for text information expressing imaging support information for supporting radiographic imaging based on the quality information, in which error information about various information in the live preview image 541 relative to the comparative optical image 551 is displayed, for example. The error information about various information includes, for example, the error (panel alignment error) of the panel alignment information in the live preview image 541 relative to the panel alignment information in the comparative optical image 551, the deviation (body axis deviation) of the body axis assistance line 542 relative to the body axis assistance line 552, and the disparity (imaging conditions disparity) of the dose in the imaging conditions of the live preview image 541 relative to the dose in the imaging conditions of the comparative optical image 551. For example, in one possible configuration, if the purpose ID of the imaging order changes, the content of the comparative optical image area 550 and the imaging support information area 560 may also be changed to information according to the purpose ID.

Also, on the imaging support screen 500 illustrated in FIG. 8, settings may be configurable not only in relation to the items to be displayed and their display positions, but also in relation to the appearance (color, font size), a warning notation, a link notation indicating association with an image, and the like, and may be switched according to the imaging purpose (purpose ID) or the like. For example, conditions may be set such that the panel alignment error and the body axis deviation are displayed only if the error or deviation is equal to or greater than a certain value, whereas the imaging conditions disparity is displayed regardless of magnitude. For example, in 10 consideration of the case where the comparative optical image area 550 is not displayed, the body axis deviation may be displayed not only as text information in the imaging support information area 560 as in FIG. 8, but also as a representation of not only the current body axis assistance line 542 but also the body axis assistance line 552 superimposed onto the live preview image area 540, the body axes may be color-coded, and the width and angle (body axis deviation) between the body axes may be shown on the image.

Next, the controller 211 changes the imaging support information being displayed on the main display 31, according to an imaging operation by the user (step S31). The imaging operation in step S31 encompasses not only operation input received from the user through the input device 32 with respect to the imaging conditions buttons 510 and the image adjustment menu area 530 on the imaging support screen 500 that serves as imaging support information, but also operations such as manual repositioning of the imaging area of the subject, the FPD 1, the tube 23, and the rounds car RC by the user, for example.

In step S31, as the user adjusts the position of the tube 23 and the like, for example, the controller 211 supports radiographic imaging by monitoring the imaging support information (for example, the live preview image 541 and the comparative optical image 551) being displayed, and displaying alert information if an alert occurs (for example, if the imaging area in the live preview image 541 differs from the imaging area in the comparative optical image 551, or if the error or deviation in the imaging support information area 560 is greater than a predetermined threshold value), assistance information assisting the user with imaging operations, and the like in real time on the main display 31.

Also, in step S31, the controller 211 may be configured to support radiographic imaging by outputting the above imaging support information, alert information, assistance information, and the like as speech (automated voice) or sound through the audio output device 33. Note that the rounds car RC may also be provided with light emitters for various colors and a vibrator in the generator main body 21, the irradiation instruction switch 22, or the like, and the controller 211 may be configured to output alert information and the like through light emission (colored light emission), vibration, and the like. Moreover, the controller 211 may also be configured to transmit the imaging support information, alert information, assistance information, and the like through the communicator 214 to a mobile terminal such as a smartphone carried by the user, thereby causing the information to be outputted (through displaying, light emission (colored light emission), or vibration) from the mobile terminal.

Conversely, in step S31, the controller 211 may be configured to determine whether the information (such as the live preview image and the alignment information) pertaining to the current radiographic imaging clears a quality assurance range based on the quality information, and notifies the user by outputting a status of clearing the quality assurance range (such as through displaying on the main display 31, the output speech or sound from the audio output device 33, light emission (colored light emission), vibration, or output from a mobile terminal).

Moreover, during radiographic imaging, the four corners of the FPD 1 must be contained within the irradiation field. However, the irradiation field lamp is difficult to see in a brightly lit room. For this reason, the rounds car RC is provided with a laser generator, and in step S31, the controller 211 may be configured to control the position of laser light from the laser generator and assist the user with visually aligning the irradiation field (FPD 1) with the laser light.

Moreover, a configuration is possible in which, in steps S30 and S31, as a rate-limiting measure for dynamic analysis of dynamic radiograph data, guide information for satisfying analysis requirements (such as a number of frames required for each of ventilatory/blood flow analysis and conditions regarding bare portion) may be displayed on the main display 31.

Also, in radiographic imaging, an aid for the subject (for example, something that is wrapped around the subject's body so that the subject moves as little as possible) may be used in some cases. Accordingly, a configuration is possible in which, in steps S30 and S31, guide information (such as positioning assistance with the aid and information for reducing influence of the aid on image analysis) for checking the irradiation field with and without the aid may be displayed on the main display 31.

Also, in step S31, the controller 211 may be configured to automatically make the SID of the laser of the rangefinder 29 conform to the imaging conditions. Also, in step S31, the controller 211 may be configured to display assistance information for measurement of the SID (such as a table of laser distances and SIDs) on the sub-display 28.

Also, in steps S30 and S31, the controller 211 may be configured to determine the degree of agreement in the motion of the subject (between a dynamic comparative optical image 551 and the live preview image 541, for example). The information about the movement of the subject to be imaged is not limited to optical image data of a live preview image, and a respiratory signal from a ventilator, thermographic data captured by a thermographic camera, and the like may also be used.

Also, in steps S30 and S31, the controller 211 may be configured to display the placement of the rounds car RC, a schematic diagram of the arrangement of the tube 23 and the FPD 1, or the like on the main display 31.

Next, the controller 211, in response to operation input related to radiographic imaging from the user through the input device 32 and the irradiation instruction switch 22, performs radiographic imaging of the subject using the FPD 1 and the radiation generator 2, and acquires captured radiograph data of the subject from the FPD 1 (step S32). Thereafter, the controller 211 associates and saves, in the storage 212, various information (such as the radiograph data, the imaging order, alignment information based on an alignment measurement of the tube 23 and the FPD 1 obtained through optical imaging by the optical imaging device 2A, the dose of the tube 23, optical image data, the EI, and the S-value) from when the radiographic imaging was performed in step S32 (step S33), and ends the navigation process.

Note that when evaluating the image quality of radiograph data according to quality information, after the radiographic imaging in step S32 of the navigation process, the controller 211 can support the evaluation of the captured radiograph data (an imaging error decision regarding the radiograph data to be described later) by performing image processing on the radiograph data and converting the radiograph data into data that is easy to compare to past radiograph data in the quality information. The image processing is, for example, region and contour extraction, time lapse, bone removal, deviation calculation, left-right determination. Moreover, depending on the evaluation of the radiograph data, the flow may not proceed to the imaging error decision by the user in the next step, and instead the controller 211 may determine the presence of imaging error automatically and proceed to an image retake step.

Additionally, the controller 211 may be configured to display at least one of the placement location of the rounds car RC, the position of the tube support 24 supporting the tube 23 of the radiation generator 2, the position and height of the tube 23, the aperture, the dose, the SID, the position of the FPD 1, the posture of the subject, and the positional relationship (angle of opposition) between the FPD 1 and the tube 23 on the main display 31 as the imaging support information to be displayed in steps S30 and S31.

After the execution of the navigation process, the user begins making a decision regarding imaging error in the radiograph data captured in the navigation process. At this time, the controller 211 of the rounds car RC (console 3) at the rounds location, when triggered by the input of an instruction for executing the decision support process from the user through the input device 32, for example, executes the decision support process by following the decision support program stored in the storage 212.

As illustrated in FIG. 10, first, the controller 211 retrieves and acquires from the storage 212 the quality information for supporting a decision regarding imaging error in the captured radiograph data corresponding to (the subject (patient) of) the imaging order selected and inputted in step S21 of the navigation process, acquires rounds imaging information including the acquired quality information and various information pertaining to the current radiographic imaging, and acquires, from the selected imaging order and the acquired rounds imaging information, patient-specific information about the subject of the rounds imaging (step S41). The rounds imaging information in step S41 is assumed to include information for performing radiographic imaging, information associated with radiograph data obtained as a result of performing radiographic imaging, and information related to the radiographic imaging apparatus. Note that the rounds imaging information may also be assumed to include at least one of information for performing radiographic imaging, information associated with radiograph data obtained as a result of performing radiographic imaging, and information related to the radiographic imaging apparatus. The patient-specific information is assumed to include at least one of disease information indicating a disease of the subject (patient) and previous imaging information that serves as various information pertaining to when radiographic imaging was performed on the subject previously.

Next, the controller 211 determines whether disease information indicating a disease of the subject to be imaged exists among the patient-specific information acquired in step S41 (step S42). If disease information exists (step S42; YES), the controller 211 acquires, from the quality information acquired in step S22, reference data (condition of disease is normal/abnormal) about other patients in the past corresponding to the disease information in step S42 (step S43). The reference data (normal/abnormal) in step S43 includes radiograph data from when radiographic imaging (normal/abnormal) was performed on other patients having the disease in the past, imaging conditions such as alignment information for rounds imaging, patient information (physique/build) about the subject, and the like.

If disease information does not exist (step S42; NO), the controller 211 acquires, from the patient-specific information acquired in step S41, generic reference data (condition of disease is normal) about other, healthy patients in the past (step S44). The reference data (normal) in step S44 includes radiograph data from when radiographic imaging (normal) was performed on other patients in the past, imaging conditions such as alignment information for rounds imaging, patient information (physique/build) about the subject, and the like.

Next, the controller 211 determines whether previous imaging information that serves as various information pertaining to when radiographic imaging was performed previously on the subject to be imaged exists among the patient-specific information acquired in step S41 (step S45). If previous imaging information exists (step S45; YES), the controller 211 acquires, from the previous imaging information in step S45, imaging conditions such as alignment information pertaining to when radiographic imaging was performed previously, radiograph data, and the like (step S46).

Next, the controller 211 retrieves and acquires, from the storage 212, various information, including the radiograph data from when radiographic imaging was performed that was stored in step S33 of the navigation process, as imaging result information (step S47).

Next, the controller 211 checks rules for selecting the radiograph data to be displayed as decision support information (step S48). The rules for selecting the decision support information in step S48 may be set in advance and stored in the storage 212, or inputted by the user through the input device 32 in step S42. The rules for selecting the decision support information include information regarding whether to prioritize (past radiograph data associated with) the same disease information as past radiograph data to be displayed together with the captured radiograph data.

Next, the controller 211 determines whether to prioritize disease information according to the rules for selecting the decision support information that were checked in step S48 (step S49). In the case of prioritizing disease information (step S49; YES), the controller 211 sets up a comparison between the reference data (normal/abnormal) regarding another patient with the same disease as the subject to be imaged that was acquired in step S43, or the generic reference data (normal) regarding another patient that was acquired in step S44, and the imaging result information pertaining to the subject to be imaged that was acquired in step S47 (step S50).

In the case of not prioritizing disease information (step S49; YES), the controller 211 sets up a comparison between the imaging conditions from the previous imaging that were acquired in step S46 and the imaging result information pertaining to the subject to be imaged that was acquired in step S47 (step S51).

Next, the controller 211 checks rules for displaying the radiograph data to be displayed as decision support information (step S52). The rules for displaying the decision support information may be set in advance and stored in the storage 212, or inputted by the user through the input device 32 in step S52. The rules for displaying the decision support information include screen information for decision support information for deciding imaging error with respect to past radiograph data (comparative image data) to be displayed together with the captured radiograph data or other imaging result information, including information regarding how to arrange and display various information, and contains a radiograph/comparative image display method (side-by-side/superimposed/sequential), the number of display frames (2/3/many), and image processing application conditions, for example.

Next, the controller 211, obeying the display rules checked in step S52, generates (screen data for) decision support information supporting a decision regarding imaging error in the radiograph data stored in step S33, the decision support information including the rounds imaging information acquired in step S41, the imaging result information including the radiograph data acquired in step S47, the reference data about the disease in question set for comparison in step S50 or the imaging conditions of the previous imaging set in step S51, and the radiograph data, and displays the generated decision support information on the main display 31 (step S53).

In step S53, the decision support screen 500 illustrated in FIG. 11 is displayed, for example. The decision support screen 500 is an example of a decision support screen with regard to imaging error in radiographic imaging that treats the chest (lung field) as the imaging area of the subject. The decision support screen 500 includes a captured radiograph area 510, a comparative radiograph area 520, a decision support information area 530, and the like.

The captured radiograph area 510 is a display area for a captured radiograph 511 based on the radiograph data of the imaging area (chest) of the subject captured through radiographic imaging by the FPD 1 and the radiation generator 2 in the navigation process. The captured radiograph 511 is a radiograph of the chest of the subject to be imaged, captured through radiographic imaging. A body axis assistance line 512 of the subject generated through image analysis of the captured radiograph 511 by the controller 211 is superimposed onto the captured radiograph 511.

The comparative radiograph area 520 is a display area for a comparative radiograph 521 based on the past radiograph data of the imaging area (chest) of another patient. A body axis assistance line 522 of the patient as the imaging subject is superimposed onto the comparative radiograph 521. Also, the captured radiograph 511 and the comparative radiograph 521 are arranged side-by-side horizontally to allow for easy visual comparison by the user. Note that if a user interface (UI) not illustrated is operated, or if a display rule is set in advance, the transparency of either the captured radiograph 511 or the comparative radiograph 521 may be adjusted such that the two images can be superimposed and displayed as a composite image.

The decision support information area 530 is a display area for text information for supporting a decision regarding imaging error in the radiograph data based on the rounds imaging information (quality information), in which error information about various information in the captured radiograph 511 relative to the comparative radiograph 521 is displayed, for example. The error information about various information includes, for example, the error (panel alignment error) of the alignment information in the captured radiograph 511 relative to the alignment information in the comparative radiograph 521, the deviation (body axis deviation) of the body axis assistance line 512 relative to the body axis assistance line 522, the disparity (difference in EIT and S-value) in a target exposure index (EIT) and S-value in the imaging conditions of the radiograph 511 relative to the EIT and S-value in the imaging conditions of the comparative radiograph 521, and a determination of prescribed information (lung field ABC determination) regarding the lung field in the comparative radiograph 521 and the captured radiograph 511. For example, if the purpose ID of the imaging order changes, the content of the comparative radiograph area 520 and the decision support information area 530 is also changed to information according to the purpose ID.

Also, on the decision support screen 500 illustrated in FIG. 11, settings may be configurable not only in relation to the items to be displayed and their display positions, but also in relation to the appearance (color, font size), a warning notation, a link notation indicating association with an image, and the like, and may be switched according to the imaging purpose (purpose ID) or the like. For example, conditions may be set such that the panel alignment error and the body axis deviation are displayed only if the error or deviation is equal to or greater than a certain value, whereas differences in the EIT and S-value are displayed regardless of magnitude. For example, in consideration of the case where the comparative radiograph area 520 is not displayed, the body axis deviation may be displayed not only as text information in the decision support information area 530 as in FIG. 11, but also as a representation of not only the current body axis but also the body axis from the previous imaging superimposed onto the captured radiograph area, the body axes may be color-coded, and the width and angle (body axis deviation) between the body axes may be shown on the image.

Note that instead of, or in addition to, the El, EIT, and S-value in the imaging conditions, the exposure index related to noise in a radiograph described in JP 2020-130796A may be used.

Also, in the example of FIG. 11, the controller 211 combines and processes the captured radiograph data and the rounds imaging information to generate the decision support screen 500 that serves as decision support information. However, the controller 211 is not limited to this configuration, and may also be configured to generate decision support information by selecting one from among using the rounds imaging information directly, processing the rounds imaging information, and combining and processing the captured radiograph data and the rounds imaging information.

As illustrated in steps S41 to S53, the controller 211 determines the type of decision support information to be generated on the basis of the rounds imaging information, and uses the rounds imaging information to generate the decision support information.

Next, the controller 211 accepts, from the user through the input device 32, the input of an imaging error decision result regarding the radiograph data, and saves the decision support information in association with the radiograph data and the imaging error decision result in the storage 212 (step S54).

Next, the controller 211 transmits the radiograph data associated with the patient ID, the imaging area ID, and the purpose ID of the imaging order to the RIS 60 through the communicator 214 (step S55). In step S56, as illustrated in FIG. 5B, the RIS 60 receives the radiograph data associated with the imaging order from the rounds car RC, saves the received radiograph data in internal storage, and transmits the received radiograph data to the terminal apparatus 50. The terminal apparatus 50 receives the radiograph data associated with the imaging order from the RIS 60 and displays the received radiograph data on its own display. The clinician observes the radiograph of the radiograph data of the subject at the rounds location displayed on the terminal apparatus 50.

Next, the controller 211 transmits the decision support information and imaging result information associated with the patient ID, the imaging area ID, and the purpose ID of the imaging order to the external apparatus 70 through the communicator 214 (step S56), and ends the decision support process. The imaging result information includes the radiograph data, and the imaging error decision result associated with the patient ID, the imaging area ID, and the purpose ID of the imaging order, various other information (such as optical image data) stored in step S33 of the navigation process, the imaging date and time, and the like. As illustrated in FIG. 5B, in step S56, the external apparatus 70 receives the decision support information and imaging result information associated with the imaging order from the rounds car RC, and stores the received information in internal storage. The imaging result information is also utilized as quality information for future imaging.

Also, the quality information which is stored in the external apparatus 70 and to be utilized for future imaging is not limited to the imaging result information, and may also be decision support information, memo information (such as notes on failed cases and important points regarding the imaging associated with the patient ID) expressing notes from the user inputted through manipulation of the input device 32, and the like. Also, the operation of storing memo information is preferably configured such that the memo information can be saved in the storage 212 and/or the external apparatus 70 simply by having the user use the input device 32 to easily check checkboxes displayed on the main display 31. Moreover, the rounds car RC may also be provided with a speech input device such as a microphone, and may be configured to accept the input of memo information as user speech through the speech input device.

Also, in the imaging result information, the radiograph data of the radiographic imaging and the optical image data captured by optical imaging for the radiographic imaging are assumed to be associated with each other. Also, in one possible configuration, the allowable range of quality information levels in the quality information may be extended according to the condition of the patient.

Also, in one possible configuration, calibration may be performed by the external apparatus 70 with respect to the quality information stored in the external apparatus 70. For example, the user consults with the clinician on the basis of the quality information stored in the external apparatus 70. The external apparatus 70 corrects the radiograph data (and/or optical image data) for reference (comparison) and various data in the stored quality information according to operation input by the user or the clinician. Outliers (noise) in the quality information are excluded. For example, radiographic imaging that had to be performed under conditions that did not meet quality standards due to circumstances such as the patient's condition, radiographic imaging as operational testing, and the like may be excludable or correctable as outliers.

Note that the information saved in the external apparatus 70 may be similarly stored in the storage 212 and re-utilized for future imaging. Depending on the capacity of the storage 212, the quality information acquired in step S13 may also be stored in an ongoing manner to reduce the acquisition processing cost of pre-acquisition for the next examination of the same patient (subject). Moreover, the external apparatus 70 may be used as portable storage for the rounds car RC in the form of externally attached semiconductor memory (such as a solid-state drive (SSD)) or an HDD. With this configuration, the transmission process from the rounds car RC to the external apparatus 70 (step S56) is omitted and the controller 211 of the rounds car RC performs only the process of saving the decision support information, imaging result information, quality information, and the like to the externally attached semiconductor memory or HDD, thereby reducing the load of a transmission process to the external apparatus 70. Furthermore, the controller 211 may also be configured to retrieve decision support information, imaging result information, and the like from an externally attached semiconductor memory or HDD, and output (transmit) the retrieved information to the external apparatus 70 at any opportunity.

At this point, rounds imaging of a neonatal patient as the subject in a neonatal intensive care unit (NICU) will be described as one practical example in relation to the navigation process. The rounds imaging information in this practical example is, for example, environment information (incubator information) about the subject, the dose of previous radiographic imaging, the previous radiograph data (optical image), and the condition (state of activity) of the subject prior to radiographic imaging. The imaging support information is, for example, a corrected dose setting (suggested information) accounting for the dose of the previous imaging and the environment information, the previous radiograph data (optical image), and suggested information regarding an imaging sequence (method) in association with the state of activity of the subject.

For a neonate, the radiation dose should be reduced by narrowing the irradiation field and the like to minimize radiation exposure. However, reproducing conditions similar to the previous imaging is difficult due to the presence or absence of a grid, the relationship between the type (transparency level) of the incubator and the imaging location (the locations of holes in the incubator), catheter state, and the like. Accordingly, a response example like the following is assumed.

### <Response Example>

Before the navigation process, the user checks the type of incubator in which the subject (neonate) has been placed. The controller 211 of the rounds car RC selects incubator information about the type of incubator being used. The controller 211 may display incubator information choices on the user interface (UI) of the main display 31 and accept input for selecting incubator information from the user through the input device 32, or if incubator information is already included in the imaging order received from the RIS 60 or the quality information received from the external apparatus 70, the controller 211 may make a determination from the included incubator information.

In steps S30 and S31, the controller 211 changes the dose setting of the tube 23 (finely adjusts the dose according to the thickness of the acrylic hood of the incubator or the like) according to the incubator type being used for the subject according to the above determination.

Alternatively, rather than before the navigation process, the controller 211 may be configured to display choices for a plurality of incubator information about the incubator being used on the main display 31 in steps S30 and S31 to allow the user to input a selection easily. Alternatively, in steps S30 and S31, the controller 211 may be configured to display choices for a plurality of incubator information about the incubator being used together with prior radiograph data captured with the same dose, thereby enabling easier selection.

Also, the navigation process may be configured such that the imaging sequence of still/dynamic images in the radiographic imaging is switched depending on whether the subject is asleep. Specifically, the user observes the sleeping state of the subject, and before the navigation process or in steps S30 and S31, the controller 211 accepts operation input from the user through a sleeping button of the input device 32, and automatically changes the content of the imaging order according to the operation input regarding whether the subject is asleep. For example, the controller 211 changes the imaging order such that radiographic imaging of a still image is performed while the subject is asleep, in consideration of little body movement, and radiographic imaging of a dynamic image is performed while the subject is awake, in consideration of body movement (conversely, a configuration that performs radiographic imaging of a dynamic image while the subject is asleep is also possible). Alternatively, the controller 211 may be configured to change the sequence of the imaging order so as to prioritize radiographic imaging of an asleep (or awake) subject.

Note that in one configuration, the quality information may be stored in the storage 212 in association with at least one of the placement location of the rounds car RC and the movement of the subject based on optical image data. In this configuration, the controller 211 analyzes the placement location of the rounds car RC and the movement of the subject from still or dynamic optical image data captured by optical imaging when performing radiographic imaging, and stores imaging result information as quality information in the external apparatus 70 in association with the analyzed placement location of the rounds car RC and the movement of the subject. Quality information corresponding to the placement location of the FPD and the movement of the subject may also serve as imaging result information when radiographic imaging is performed by a different rounds car than the rounds car RC. According to this configuration, for example, the controller 211 can display quality information (such as past optical image data) with the same placement location of the rounds car RC and movement of the subject as in the current radiographic imaging on the main display 31 as imaging support information, thereby supporting radiographic imaging in which the placement location of the rounds car RC and the movement of the subject are the same, and raising the image quality of the radiograph data acquired in the current case.

Next, FIG. 12 will be referenced to describe a practical example of the decision support process. The subject is a patient admitted for a pulmonary disease, and is a case in which radiographic imaging is performed while making the rounds of a ward (for example, rounds imaging in a coronavirus ward). When radiographic imaging is performed during a deep breath (inhalation state), the lung field in the radiograph is smaller (the position of the diaphragm is higher) than expected, and in some cases, the user may have difficulty deciding whether to do an image retake according to whether the subject is not breathing correctly. The rounds imaging information is, for example, radiograph data from the previous imaging, radiograph data (normal/abnormal) for the same case of disease, and similar radiograph data. The decision support information is, for example, comparative radiograph data to be displayed side-by-side and an assistance line indicating the position of the diaphragm.

In step S53 of the decision support process, the decision support screen 600 illustrated in FIG. 12 is displayed on the main display 31, for example. The decision support screen 600 includes a patient information area 610, select buttons 620, a captured radiograph area 630, and comparative radiograph areas 640 and 650.

The patient information area 610 is a display area for patient information (patient ID, patient name, sex, date of birth, requesting department) based on the imaging order. The select buttons 620 are buttons for accepting the input of the imaging area and the type of image data (still image, dynamic image). In this case, for example, it is possible to switch between a “thoracic moving image PA” and an “abdominal moving image PA”.

The captured radiograph area 630 is a display area for a captured radiograph 631 based on the radiograph data of the imaging area (chest) of the subject captured through radiographic imaging by the FPD 1 and the radiation generator 2 in the navigation process. The captured radiograph 631 is a radiograph of the chest (thoracic moving image PA) of the subject to be imaged, captured through radiographic imaging. An assistance line 632 indicating the position of the diaphragm of the subject generated through image analysis of the captured radiograph 631 by the controller 211 is superimposed onto the captured radiograph 631.

The comparative radiograph area 640 is a display area for a comparative radiograph 641 based on the past radiograph data of the imaging area (chest) of another (healthy) patient whose condition is normal. An assistance line 642 indicating the position of the diaphragm of the patient as the imaging subject is superimposed onto the comparative radiograph 641. The comparative radiograph area 650 is a display area for a comparative radiograph 651 based on the past radiograph data of the imaging area (chest) of another patient whose condition is abnormal (for example, excess visceral fat). An assistance line 652 indicating the position of the diaphragm of the patient as the imaging subject is superimposed onto the comparative radiograph 651. Also, the captured radiograph 631 is arranged side-by-side horizontally with the comparative radiographs 641 and 651 to allow for easy visual comparison by the user.

The trailing edge of the 11th rib is emphasized (with a black line, for example) by the assistance lines 632, 642, and 652 as a decision criterion regarding the position of the diaphragm. Also, in one configuration, the position of the lower lung (lower lobe), a decrease in transparency is easily seen, may be enlarged by default enlargeable by manipulation of the input device 32. On the other hand, if there are no findings, such as in an emergency or first-timing imaging, identification is not possible and therefore the display may be limited to reference information. For example, the abnormal comparative radiograph 651 may be a reference image of interstitial pneumonia or an example of reduced diaphragmatic movement due to high levels of visceral fat.

Also, on the decision support screen 600 in FIG. 12, the lines of the assistance lines 632, 642, and 652 are drawn to indicate the position of the diaphragm, but points to check may also be enclosed with a circle or a rectangle, pointed out by an arrow, or expressed by a message indicating the diaphragm. In this case, visibility of the object of comparison may be increased by displaying a shape, symbol, or text indicating the same object not only in the captured radiograph area 630 but also in a past image or a reference image serving as a comparative radiograph (comparative radiograph areas 640, 650). Alternatively, the shape or color may be partially altered to enable the user to distinguish which drawn portion indicates abnormality and which indicates normality (for example, a normal sample is indicated by a blue dashed line while an abnormal sample is indicated by a red dashed line, and on the captured radiograph, a solid line is displayed with the color closer to red or blue so that the threshold determination can be understood at a glance).

Next, a different practical example of the imaging decision process will be described. The following is a case of follow-up observation of a subject who was taken to the medical facility after a traffic accident and diagnosed with a broken rib. The rounds imaging information is, for example, information about the requesting department/requesting physician, information about the bed angle/alignment during radiographic imaging, and the above information when radiographic imaging was performed previously. The decision support information is, for example, information (for example, rotator cuff width) pertaining to a designed region of interest (ROI) in a radiograph, a threshold value comparison result with respect to the value of the rotator cuff width, and a tolerance range correction associated with the imaging environment and/or the diagnosing physician.

Points to note when performing radiographic imaging and interpreting the radiograph are defined for each medical facility as decision criteria for fractures, tendon ruptures, and the like. In such cases, whether the information necessary for the clinician to make a diagnosis has been captured must also be considered to determine the success or failure of radiographic imaging.

### <Response Example>

For example, in the case where a ray-summation (RaySum (RS)) process for determining whether the rotator cuff width is less than or equal to 5 mm has been added, the controller 211 does not simply indicate whether the rotator cuff width is less than or equal to 5 mm, but combines this information with information about the bed angle and alignment when radiographic imaging was performed, and displays this information together with an indication of whether to apply a measured value correction.

In addition, the controller 211 changes the tolerance range of the irradiation angle (alignment) for patient imaging, or displays both a normal tolerance range and a reference tolerance range according to the clinician as ancillary information, depending on clinician information (such as request information, or simply the name of the clinician who placed the imaging order). For example, information is displayed to indicate that clinician A1 was able to diagnose the patient previously even though the bed was slightly inclined and deviated about 5° from orthogonal due to the physical condition of the patient, whereas clinician B1 deemed such deviation unsuitable and ordered an image retake (specifically, the irradiation angle in imaging error or successful imaging in the past is displayed or used as a threshold value for error determination, depending on the clinician who ordered imaging).

Next, a different practical example of the imaging decision process will be described. The following is a case of post-operative observation of a surgery patient with an acute myocardial infarction as the subject. The rounds imaging information is, for example, tube presence information, an image analysis result regarding the captured image (such as a determination of whether tubes are present), notes from the user (radiographer) regarding the rounds imaging, the imaging conditions of the previous radiographic imaging, past captured image data from a time when no tubes were present, and measurement result information from an electrocardiogram or a pulse oximeter. The decision support information is, for example, analysis image data with tubes removed relative to captured radiograph data, past radiograph data from a time when no tubes were present, notes from the user regarding the rounds imaging, and measurement result information from an electrocardiogram or a pulse oximeter.

Cardiac motion evaluation (ventricular wall motion evaluation) is performed using a dynamic rounds car RC as a simplified alternative to organ catheterization. For example, the heart's rate of expansion and contraction is calculated and checked to see if it stays equal to or greater than a certain numerical value. In such cases, if the subject is given extracorporeal membrane oxygenation (ECMO) or drains, it is unclear whether the imaging area is being captured correctly by radiographic imaging due to the tubes.

### <Response Example>

The controller 211 ascertains the position of tubes from image analysis of the radiograph data or advance information (tubes Y/N). Accordingly, the controller 211 displays a past radiograph without tubes, a radiograph that has been subjected to a tube removal analysis process, or the like as a comparative radiograph with respect to radiograph data captured through normal imaging. The tube removal analysis process involves image interpolation at the tube position between successive frames, for instance.

Additionally, the controller 211 saves the previous imaging conditions (placement location of the rounds car, SID, alignment information, dose) and user comments (precautions, notes) in association with each other as a set in the storage 212 and the external apparatus 70. The previous imaging conditions and user comments are displayable on the imaging screen prior to radiographic imaging, for example. Also, if the imaging location is close to a bed, a bedside monitor or the like may also be utilized to additionally display measurement result information from an electrocardiogram or a pulse oximeter, for instance.

At this point, the content of the decision support information will be summarized. The decision support information may include the radiograph data of the current imaging and, as the comparative radiograph data to be compared to the radiograph data of the current imaging, past radiograph data of the subject to be imaged/another patient (normal (healthy (radiograph as a generic example)/abnormal condition)). The comparative radiograph data may also be pre-operative radiograph data for a surgery.

Also, the decision support information may include comparative information with respect to the previous or other past radiographic imaging result. The comparative information is, for example, the imaging range, position, direction, specific ROI (the range, size (area), position, direction, pixel value, and time variation thereof), information indicating a positional relationship with respect to a specific structure (body axis, ribs, diaphragm, rib cage, immobile structure), radiation irradiation conditions, irradiation results (mAs value, irradiation time, SID, EIT, S-value, angle of tube 23, alignment information, aperture of collimator 25, grid use Y/N), the imaging method (imaging pose of subject, breathing method, aid Y/N, gravity method), and the state of the subject (estimated body thickness, imaging time (time since last meal), accessories Y/N (such as a ventilator or tubes), conscious/unconscious, body movement Y/N) with regard to the previous imaging/current imaging.

Also, the decision support information may include comparative information of before and after a specific process is performed. The specific process is the attachment or removal of a ventilator, ECMO, or the like, suture removal, or gauze removal, for instance. Moreover, the decision support information may include information pertaining to the temporal change in a plurality of past radiograph data. The information pertaining to the temporal change is, for example, comparative information regarding the magnitude of a change between “the last imaging and the present” or “the second-to-last imaging and the last imaging”, and comparative information on a yearly level (such as the same day one year ago and the same day two years ago) as follow-up observation.

Also, the decision support information may include presentation information indicating the presence or absence of a grid or whether a scattering correction process is necessary. For example, even if scattering correction is off by default, in imaging cases where turning on scattering correction would be helpful for decision support, the presentation information is information prompting the user to turn on the scattering correction, or even if the grid is on, if performing an additional scattering correction process would result in image quality providing decision assistance, the presentation information is information prompting the user to turn on the scattering correction process.

Also, the decision support information may include presentation information regarding a case linked to imaging failure (imaging error). For example, the presentation information is confirmation information regarding whether the radiograph matches a failure example after radiographic imaging, a dose adjustment according to the body thickness of the subject, or the irradiation angle in a formed image.

Also, the decision support information may include user (radiographer) log information. For example, the log information is notes with respect to the same patient, points to note about the previous imaging, and points to note about the specific rounds car in use.

Also, the decision support information may include information about the requesting/receiving clinician. For example, the clinician information is a request by the clinician regarding image quality, satisfaction with the level of perfection, requests or comments regarding a radiograph captured in the past, diagnostic points (such as image processing used to make those points easier to see), and the demanded level of alignment.

Also, the decision support information may include other examination information about the subject to be imaged. The other examination information is respiratory state information from the oxygen concentration according to a pulse oximeter or the like and electrocardiogram information (such as resting state or stress test).

Next, patterns of decision support information display methods relating to how the decision support information is presented to the user will be described. The controller 211 may be configured to display the decision support information superimposed onto a radiograph of the radiograph data, for example, or display the decision support information separately (side-by-side/individually/switching) from the radiograph, like on the decision support screens 500 and 600.

Additionally, the controller 211 may be configured to display radiograph data with simple switching as the decision support information limited to image retake decision points according to a hanging protocol (sequential display). For example, with the push of a button, spots determined to contain imaging error in erroneous radiograph data from the past may be sequentially switched (enlarged/reduced and subjected to image processing) and displayed. Also, when displaying the radiograph data of a still image, the point of scrutiny may be switched 10 through enlarged display of the ROI, whereas when displaying the radiograph data of a dynamic image, frames to be determined may be displayed sequentially. For example, the display may be switched in a progression like chest: full view→body movement→imaging error determination (whether rib cage, diaphragm, or the like should be visible)→candidate case (appropriate dose for ROI). Additionally, the decision support information may be displayed or not according to the switching display. For example, the decision support information may be 15 displayed in a form that is suitable for the display area, such as by displaying an assistance line indicating the position of the diaphragm in the case of a full view, not displaying an assistance line when determining body movement, and displaying assistance lines surrounding an area that should not be visible when determining visibility.

Also, as a hanging protocol, radiograph data may also be sequentially switched in combination with result information from simple measurements. For example, the display may be switched in the progression of ankle: full view→joint angle measurement→area of bone overlap→distance of specific spot. In one configuration, the switching sequence of the radiograph data may be settable in advance by user, by imaging area, and/or by imaging conditions. Also, for cases in which a switching display is unnecessary (such as cases with no body movement), a shortcut to a display sequence of a switching display may be provided.

The controller 211 may also be configured to show, as the decision support information, a juxtaposed or differential display of numerical information from the imaging conditions and irradiation conditions or imaging results for the captured radiograph data and comparative radiograph data from the past.

The controller 211 may also set whether or not the decision support information in the initial display needs to be enlarged and displayed according to conditions (ward, operation/display items). The controller 211 may also be configured to select and display an initial display image suitable for the imaging conditions and the purpose of diagnosis. For example, after confirming the need for a wipe or preview, the radiograph data captured as the real image may suddenly be displayed. More specifically, if the purpose of the operation is to check for any remaining gauze, the wipe/preview is given priority, whereas if the purpose of imaging is to make an initial diagnosis in an emergency, when connecting the rounds car RC to an external monitor, or the like, the clinician in charge will check the image immediately, and therefore the real image display enabling diagnosis is set as the initial display. Moreover, the captured radiograph data may also be displayed together with the decision support information as a set. For example, in operations or the like where a decision by the user is not required, the decision support information is deferred to hasten the initial display of the radiograph only. On the other hand, in a neonatal intensive care unit (NICU) or the like, since the neonatal subject is small and positioning is difficult, radiograph data with an enlarged ROI is displayed together with the decision support information as a set from the beginning.

The controller 211 may also be configured to display the decision support information in the initial display of a wipe/preview or limit the display of the decision support information, depending on where the decision support information is to be displayed (such as the main display 31, the sub-display 28, a bedside monitor, or an external monitor). Moreover, the controller 211 may be configured to categorize the decision support information to be displayed on the main display 31, the sub-display 28, and a bedside monitor or external monitor (such as by utilizing the bedside monitor or external monitor to display other examination information about the radiographic imaging, for example).

Also, the controller 211 may be configured to change a setting (according to operation input via the input device 32) regarding whether the display of the decision support information is required according to the ease of visual decision-making. The controller 211 may also be configured to display the decision support information with a time delay according to the priority of the decision support information.

Also, in the case of dynamic radiographic imaging, the controller 211 may be configured to display only a specific frame or frames of the captured dynamic radiograph data as the decision support information. The specific frame or frames may be a single frame suitable for determining imaging error or a group of frames in a range necessary for determining imaging error. From the comparative image data serving as the decision support information, a specific frame or frames in the same range relative to the specific frame or frames of the captured dynamic radiograph data are displayed.

Also, the controller 211 may be configured to extract two or more frames (two or more ranges) for comparison from a single dynamic image of a single set of captured dynamic radiograph data, and display the extracted frames as the comparative image data of the decision support information. The frames to be extracted are, for example, the frames of maximum exhalation/maximum inhalation, the frames of maximum/minimum pixel value within a specific ROI range, the frames of highest/lowest position of the diaphragm, or frames of an extended/contracted joint.

Also, the controller 211 may be configured to display location-specifying information with an overlay or indicator, a heatmap, or the like as the decision support information. The location-specifying information may also be combined with numerical information, and may also link to a graph or the like in response to a selection operation.

Also, the controller 211 may be configured to display an enlarged view of a point of scrutiny (ROI) in the captured radiograph data as the decision support information. The enlarged view is performed, for example, from the time of the initial display, in association with a preset or the detection of a point to pay attention to, when the imaging order from the clinician specifies a point of scrutiny, when there is a point that tends to be determined as imaging error according to the imaging area or imaging conditions, when a point of scrutiny is chosen in combination with deep learning, or when a range (frames) with an abnormality in a specified spot is initially displayed (such as pixel abnormalities or the case of unwanted visibility) in addition to a specified spot.

Also, for captured radiograph data and comparative radiograph data from the past to be displayed, the controller 211 may be configured to display, as decision support information, the reason why the radiograph data was chosen. The reason why a frame or range (for example, an enlarged ROI) in the captured radiograph data and the comparative image data from the past was chosen is displayed on or outside the images, thereby clarifying the reason for the choice and enabling the user to easily understand the locations that should be reviewed. In particular, by displaying the reason why the object of comparison (comparative radiograph data) was chosen in association with the points that should be reviewed, the significance of the comparison for making an imaging error decision by the user is improved.

Also, the controller 211 may be configured to limit the display of the decision support information to imaging error decision support items. For example, in one configuration, imaging error decision support items may be set by imaging area or imaging conditions, imaging error decision support items may be set for each user (preference/level), and radiograph data within a diagnosable range (a range in which the user can recognize imaging error at a glance) may be excluded from the decision support process and the decision support process may be limited to only those portions where the user is unable to make a decision, thereby streamlining the decision support process.

Also, the controller 211 may be configured to display the decision support information on a display other than the main display 31. For example, the following cases (1) and (2) are examples of displaying the decision support information on a display other than the main display 31.
(1). Case of displaying decision support information on sub-display 28

For example, in the case in which the radiographer (user) performs imaging while assisting the subject with their posture, the decision support information is displayed on the sub-display 28 if the user is unable to come back around to (unable to check) the console 3 immediately after imaging. More specifically, in this case, a footswitch is utilized to use the sub-display 28 to perform a basic check for imaging error easily, even if the user is near the subject (a configuration in which the sub-display 28 is close to the tube 23 like in FIG. 1).
(2). Case of displaying decision support information on an external monitor (not illustrated)

The decision support information is displayed on an external monitor in an emergency or an operation in which the decision support information is hard to see on the main display 31 of the console 3 or the small screen of the sub-display 28, or in which multiple clinicians or surgeons view the decision support information. This situation is subdivided further into the following two cases (2)-1 and (2)-2.
(2)-1. Case of hiding portion of decision support information

Since the display information on the external monitor is also visible to the subject, a quality determination result such as an imaging error decision among the decision support information is hidden to avoid causing anxiety.
(2)-2. Case of displaying decision support information

In this case, the display of a quality determination result is prioritized (emphasized) to keep the radiograph from being judged as a failed image by a surgeon or clinician.

Also, the controller 211 may be configured to emphasize the decision support information. An emphasized display is, for example, emphasis using color/thickness/blinking of a mark or an overlay on a frame or ROI of the radiograph data and/or comparative image data, emphasis using color/thickness/blinking of a frame border, mandatory confirmation using a message display (dialog display or a modal window (a window that disallows other operations until the window is closed)), and a display with an intensity applied according to the priority of the information to be seen. Instead of a display, a message may also be outputted as sound (including speech) by the audio output device 33, or as light output or vibration output if the rounds car RC (such as the generator main body 21 or irradiation instruction switch 22) includes a light source or a vibrator), for instance.

According to the present embodiment as above, the radiographic imaging system 100 that serves as a radiographic imaging apparatus is provided with: the radiation generator 2 that serves as a radiation emitter that irradiates a subject treated as the imaging subject with radiation; the FPD 1 that serves as a detector that performs radiographic imaging of the subject by detecting radiation and generates radiograph data; and the controller 211 that acquires an imaging order related to radiographic imaging of the subject, acquires, from an external apparatus, quality information for evaluating the image quality according to the imaging purpose of the radiographic imaging based on the imaging order, and stores the acquired quality information in the storage 212.

Accordingly, with the quality information, the user (which may be in particular a user unfamiliar with rounds imaging) can evaluate the image quality of the radiograph data according to the imaging purpose at the rounds location. More specifically, in the imaging support process, the quality information can be used to support the radiographic imaging and indirectly evaluate and enhance the image quality of the radiograph data to be captured. In the decision support process, the quality information can be used to support a decision regarding imaging error in the radiograph data to evaluate the image quality, and enhance the image quality of radiograph data without imaging error.

Also, the controller 211 performs desired image processing on the radiograph data, and generates radiograph data that can be compared to past radiograph data in the quality information. Consequently, in the decision support process, the radiograph data subjected to image processing and the radiograph data in the quality information can be compared, a more appropriate decision regarding imaging error in the radiograph data can be made, and the image quality of radiograph data without imaging error can be enhanced.

The controller 211 also acquires quality information from before the rounds car RC and the FPD 1 are moved to the rounds location where the subject is present, or before the radiographic imaging is performed. For this reason, the quality information can be acquired reliably before the radiographic imaging (navigation process, decision support process).

Also, the imaging order includes a purpose ID that identifies the imaging purpose. The controller 211 acquires quality information corresponding to the purpose ID. For this reason, quality information according to the imaging purpose can be acquired easily using the purpose ID.

Note that the storage that stores the quality information may also be storage separate from the rounds car RC (such as storage in an external apparatus that can communicate with the rounds car RC at the rounds location, or external storage that attaches to the rounds car RC), rather than the storage 212. According to this configuration, the capacity of the storage 212 can be lowered and the configuration of the rounds car RC can be simplified.

In addition, according to the present embodiment from a different aspect, the radiographic imaging system 100 (rounds car RC) that serves as a radiographic imaging apparatus is provided with the controller 211 that acquires rounds imaging information related to the radiographic imaging of the subject treated as the imaging subject when making rounds, and outputs, on the basis of the acquired rounds imaging information, decision support information supporting a decision regarding whether the radiograph data generated by the radiographic imaging satisfies a prescribed image quality.

Accordingly, with the decision support information, after performing rounds imaging, the user can decide whether the radiograph data satisfies the prescribed image quality and ensure the image quality level of the radiograph data.

Also, the controller 211 determines the type of decision support information to be generated on the basis of the rounds imaging information. For this reason, decision support information of the appropriate type can be generated.

Also, the controller 211 uses the rounds imaging information to generate the decision support information. For this reason, appropriate decision support information can be generated.

Also, the controller 211 generates decision support information by selecting one from among using the rounds imaging information directly, processing the rounds imaging information, and combining and processing the radiograph data of the radiographic imaging and the rounds imaging information. For this reason, decision support information can be generated by an appropriate method.

Also, the rounds imaging information includes at least one of information for performing radiographic imaging, information associated with the radiograph data obtained as a result of performing the radiographic imaging, and information related to the radiographic imaging apparatus. For this reason, appropriate decision support information can be generated.

Also, the decision support information is support information for making a decision regarding whether image retake due to imaging error in the radiograph data is necessary. Accordingly, after performing rounds imaging, the user can make an appropriate decision regarding whether image retake due to imaging error is necessary, and thereby ensure the image quality level of the radiograph data.

Also, the imaging support information includes radiograph data of the subject in the past or of another subject. In this case, the controller 211 displays the radiograph data of the subject acquired in the current radiographic imaging and radiograph data of the subject in the past or of another subject in association with each other (for example, arranged horizontally). Accordingly, the user can easily and accurately compare a radiograph from the current radiographic imaging and a radiograph of the subject in the past or of another subject, decide whether the radiograph data satisfies a prescribed image quality according to the comparison, and ensure the image quality level of the radiograph data.

Also, the radiograph data of the subject in the past or of another subject includes radiograph data of normal or abnormal disease condition in the past. Accordingly, the user can easily and accurately compare a radiograph from the current radiographic imaging and a radiograph of normal or abnormal disease condition in the past, decide whether the radiograph data satisfies a prescribed image quality according to the comparison, and ensure the image quality level of the radiograph data.

Also, the controller 211 superimposes, onto the radiograph data, assistance lines 632, 642, and 652 that serve as assistance images supporting decision-making. Accordingly, with the assistance lines 632, 642, and 652, the user can make an appropriate decision regarding whether the radiograph data satisfies the prescribed image quality and ensure the image quality level of the radiograph data.

Also, according to the present embodiment from a navigation aspect, the radiographic imaging system 100 that serves as a radiographic imaging apparatus is provided with: the radiation generator 2 that serves as a radiation emitter that irradiates a subject treated as the imaging subject with radiation; the FPD 1 that serves as a detector that generates radiograph data of the subject S by detecting radiation; the storage 212 storing quality information related to the image quality of the radiograph data; and the controller 211 that supports radiographic imaging on the basis of the quality information.

Therefore, through radiographic imaging support, radiographic imaging can be performed at the rounds location to obtain the image quality sought by the clinician, regardless of the skill and experience of the user, thereby preventing wasted time due to image retake in the case of performing radiographic imaging not of the image quality sought by the clinician, and preventing the burden of radiation exposure to the subject due to image retake.

Also, the quality information is information related to an imaging method for radiographic imaging, based on at least one of the imaging area, the subject, the user, and the clinician. Accordingly, radiographic imaging of high image quality can be performed according to a suitable imaging method corresponding to at least one of the imaging area, the subject, the user, and the clinician.

Also, the radiographic imaging system 100 (rounds car RC) is provided with the optical imaging device 2A that serves as an imaging device that generates optical image data by optically imaging the imaging subject. The quality information is stored in the storage 212 in association with at least one of the placement location of the rounds car RC and the movement of the subject based on optical image data. Accordingly, for example, the controller 211 can display quality information (such as past optical image data) with the same placement location of the rounds car RC and movement of the subject as in the current radiographic imaging on the main display 31 as imaging support information, and thereby perform radiographic imaging of high image quality in which the placement location of the rounds car RC and the movement of the subject are the same.

Also, the controller 211 generates imaging support information for supporting radiographic imaging on the basis of the quality information stored in the storage 212, and displays the imaging support information on the main display 31. Accordingly, the user can perform radiographic imaging of high image quality by looking at the imaging support information.

Also, the imaging support information includes optical image data of the subject in the past or of another subject. In this case, the controller 211 displays the optical image data of the subject acquired in the current radiographic imaging and optical image data of the subject in the past or of another subject in association with each other (for example, arranged horizontally). Accordingly, the current optical image and an optical image of normal or abnormal disease condition in the past can be compared easily and accurately, and radiographic imaging of high image quality can be performed according to the comparison.

Also, the optical image data of the subject in the past or of another subject includes optical image data of normal or abnormal disease condition in the past. Accordingly, the current optical image and an optical image of normal or abnormal disease condition in the past can be compared easily and accurately, and radiographic imaging of high image quality can be performed according to the comparison.

Also, the controller 211 displays, on the basis of the quality information stored in the storage 212, at least one of the placement location of the rounds car RC, the position of the supports 241 and 242 supporting the tube 23, the position and height of the tube 23, the aperture of the collimator 25, the dose, the SID, the position of the FPD 1, the posture of the subject, and the positional relationship between the FPD 1 and the tube 23 on the main display 31 as the imaging support information. Accordingly, by looking at the imaging support information, the user can set the radiographic imaging by the radiographic imaging system 100 into an appropriate state and perform radiographic imaging of high image quality.

Also, the controller 211 supports radiographic imaging by outputting the imaging support information and the like as speech (or sound) through the audio output device 33. Accordingly, even if the user is not in a position to see the main display 31, the user can confirm the imaging support information and the like aurally and perform radiographic imaging of high image quality.

### Modification

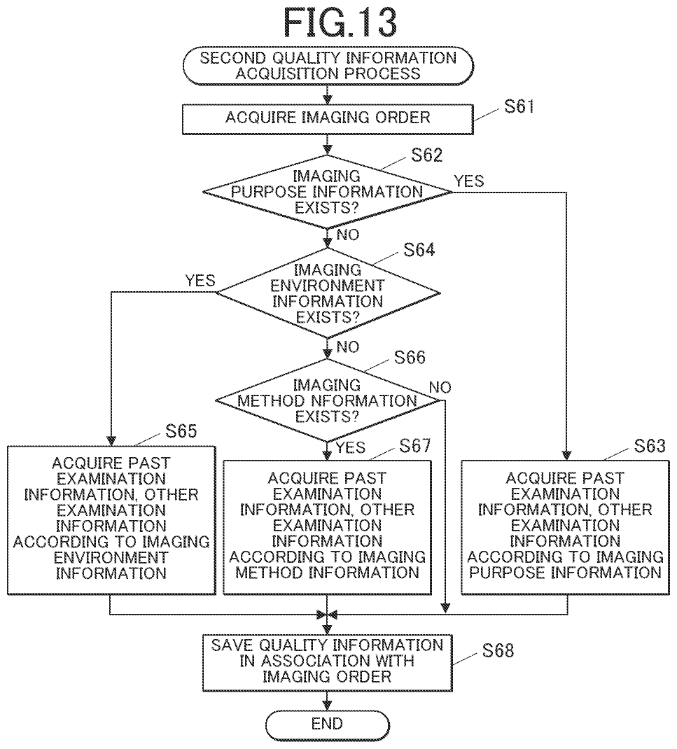

FIG. 13 will be referenced to describe a modification of the above embodiment. FIG. 13 is a flowchart illustrating a second quality information acquisition process.

In the above embodiment, the rounds car RC acquires quality information using the purpose ID, but in the present modification, the rounds car RC determines the quality information to be acquired from the content of the radiographic imaging in the imaging order. For this reason, the present modification assumes that the purpose ID is not included in the imaging order. Moreover, it is assumed that the imaging order includes at least one of imaging purpose information related to the purpose of radiographic imaging, imaging environment information related to the imaging environment of radiographic imaging, and imaging method information related to the imaging method of radiographic imaging as the information regarding the content of the radiographic imaging.

The device configuration in the present modification uses the radiographic imaging system 100, similarly to the above embodiment. However, the storage 212 of the rounds car RC is assumed to store a second quality information acquisition program for executing a second quality information acquisition process described later instead of the first quality information acquisition program, and is also assumed to store the imaging purpose table 400.

Next, FIG. 13 will be referenced to describe operations by the radiographic imaging system 100. Similarly to the above embodiment, in the case of performing radiographic imaging of the subject at the rounds location, the controller 211 of the rounds car RC (console 3) at the standby location before moving, when triggered by the start of receiving an imaging order from the RIS 60 through the communicator 214, for example, executes the second quality information acquisition process by following the second quality information acquisition program stored in the storage 212.

As illustrated in FIG. 13, step S61 is similar to step S11 of the first quality information acquisition process in FIG. 6 of the above embodiment. Next, the controller 211 determines whether imaging purpose information exists in the imaging order acquired in step S61 (step S62). If imaging purpose information exists (step S62; YES), the controller 211 determines past examination information (for example, past radiograph data and optical image data) related to a past examination of the same subject to be acquired, other examination information (for example, past radiograph data and optical image data) related to a past examination of another subject, and the like as quality information according to the imaging purpose information, transmits, to the external apparatus 70 through the communicator 214, a request for the determined quality information, including the patient ID and the imaging area ID, and receives and acquires the quality information from the external apparatus 70 (step S63).

If imaging purpose information does not exist (step S62; NO), the controller 211 determines whether imaging environment information exists in the imaging order acquired in step S61 (step S64). If imaging environment information exists (step S64; YES), the controller 211 determines past examination information related to a past examination of the same subject to be acquired, other examination information related to a past examination of another subject, and the like as quality information according to the imaging environment information, transmits, to the external apparatus 70 through the communicator 214, a request for the determined quality information, including the patient ID and the imaging area ID, and receives and acquires the quality information from the external apparatus 70 (step S65).

If imaging environment information does not exist (step S64; NO), the controller 211 determines whether imaging method information exists in the imaging order acquired in step S61 (step S66). If imaging method information exists (step S66; YES), the controller 211 determines past examination information related to a past examination of the same subject to be acquired, other examination information related to a past examination of another subject, and the like as quality information according to the imaging method information, transmits, to the external apparatus 70 through the communicator 214, a request for the determined quality information, including the patient ID and the imaging area ID, and receives and acquires the quality information from the external apparatus 70 (step S67).

In response to step S63, S65, or S67, the external apparatus 70 receives the request for quality information from the rounds car RC, retrieves from internal storage the quality information corresponding to the patient ID and the imaging area ID included in the request for quality information, and transmits the retrieved quality information to the rounds car RC. Thereafter, the controller 211 saves the quality information acquired in step S63, S65, or S67 in association with the imaging order in the storage 212 (step S68), and ends the second quality information acquisition process.

An example of the imaging environment information, imaging purpose information, rounds imaging information, and decision support information in the present modification is illustrated in the following Table II. The “accessories” in Table II refer to medical engineering (ME) equipment installed in or near the subject's body, such as a pacemaker, an implantable cardioverter defibrillator, or a pump catheter for assisted circulation.

TABLE 2
Table II

| Imaging environment information | Imaging purpose information | Rounds imaging information | Decision support information |
|---|---|---|---|
| Ward rounds | Follow-up observation | Patient information | Positioning determination |
| | | (symptom/disturbance | (position/angle/defects/area |
| | | information), case information | determination/ROI), |
| | | (points to check, ROI), imaging | image comparison |
| | | information (previous | (specific area comparison/time |
| | | comparison/alignment/grid Y/N), | lapse), graph display, |
| | | comparative image data (past | other examination results |
| | | image/case image), other | |
| | | examination information | |
| | | (ECG/pulse oximeter) | |
| Ward rounds | Post-operative follow-up | Patient information | Positioning determination |
| | | (symptom/disturbance | (area of concern (ROI)), image |
| | | information), case information | comparison (pre-op/post-op), |
| | | (points of concern) comparative | image quality determination |
| | | image data (pre-op imaging/case | |
| | | image), clinician instructions | |
| Operation | Post-operative check | Patient information | Positioning determination |
| | | (symptom/disturbance | (area of concern (ROI)), |
| | | information), case information | image comparison |
| | | (purpose of surgery), comparative | (pre-op/post-op). disturbance |
| | | image data (pre-op imaging/case image) | check (tube/catheter/gauze) |
| Emergency | Screening | Clinician instructions, | Positioning determination |
| | | comparative image data | (position/angle/defects/area |
| | | (generic image) | determination), image comparison |
| | | | (generic comparison) |
| Other | Patient individuality | Patient information | Image quality determination |
| | (body thickness) | (body thickness), imaging | (insufficient dose/pixel defects) |
| | | information (dose information) | |
| Other | Patient individuality | Patient information | Image quality determination |
| | (accessories) | (metal presence/location | (presence of artifacts, etc.) |
| | | information), imaging information | |
| | | (dose information), comparative | |
| | | image data (past image) | |
| Other | Maintenance | Hardware information | Image comparison |
| | | (voltage attenuation/years in | (change over time), graph display |
| | | service/collimator) | (change over time), image quality |
| | | | determination (insufficient dose) |

Also, an example of the imaging area, imaging method information, rounds imaging information, and decision support information in the present modification is illustrated in the following Table III.

TABLE 3
Table III

| Imaging area | Imaging method information | Rounds imaging information | Decision support information |
|---|---|---|---|
| Joint | Weighted imaging | ROI information, case | Positioning (e.g., in knee case, |
| (knee joint, | | information to be checked | lateral/medial malleolus |
| elbow joint, | | (direction/angle), weighting | overlap/left-right |
| ankle joint . . .) | | direction (previous) | mistake/wrong area), |
| | | | weighting direction |
| Joint | Dynamic image | ROI information, case | Positioning (e.g., in knee case, |
| (knee joint, | (mobile) | information to be checked | lateral/medial malleolus |
| elbow joint, | | (direction/angle), previous | overlap/left-right |
| ankle joint . . .) | | range, previous time | mistake/wrong area), |
| | | | difference from previous |
| Limb | Normal imaging | Comparative information | Area left-right mistake, |
| (arm, leg) | | (normal image), ROI | wrong area |
| | | information, case information | |
| | | to be checked (area in imaging | |
| | | conditions), weighting | |
| | | direction (previous) | |
| Limb | Dynamic image | Comparative information | Area left-right mistake, |
| (arm, leg) | (mobile) | (normal image), ROI | wrong area, difference |
| | | information, case information | from previous |
| | | to be checked (area in imaging | |
| | | conditions), previous range, | |
| | | previous time | |
| Torso | Normal imaging | ROI information, optimal dose | Saturation due to |
| (abdomen, spine, | | setting information by area, | overdose, body |
| hip joint) | | area information (body thickness) | movement, wrong area |
| Chest | Normal imaging | Image data to be compared | Missing lung field, |
| | | (past image/clinical image), | wrong area, lung field |
| | | ROI information, case | region shape, position, |
| | | information to be checked, | size, inclination |
| | | selected panel information | |
| | | (size/direction) | |
| Chest | Stress imaging | Image data to be compared | Missing lung field, |
| | | (past image/clinical image), | wrong area, lung field |
| | | ROI information, case | region shape, ECG |
| | | information to be checked, | information, difference |
| | | selected panel information | from resting state |
| | | (size/direction), stress | |
| | | information, ECG results | |

The image data in Tables II and III is radiograph data, but may also encompass optical image data.

Note that the priority of determining (order in which to determine) the presence or absence of imaging purpose information, imaging environment information, and imaging method information in the imaging order is not limited to the example of the second quality information acquisition process in FIG. 13. Moreover, the priority of determining the presence or absence of imaging purpose information, imaging environment information, and imaging method information may be set by the medical facility, the user, the medical department, or the like.

Also, the determination of the presence or absence of imaging purpose information, imaging environment information, and imaging method information in the imaging order is not limited to the example of the second quality information acquisition process in FIG. 13. The determination of the presence or absence of imaging purpose information, imaging environment information, and imaging method information in the imaging order may be a combination of any two of the above, or any one of the above.

Also, the quality information to be acquired may be determined simply according to the acquired imaging order, or operation input regarding the content of quality information to be added may be accepted from the user through the input device 32, or operation input for displaying, on the main display 31, choices for the content of quality information to be added and selecting a choice may be accepted, and the quality information to be acquired may be determined according to the operation information.

Also, the type of quality information to be acquired is not limited to quality information based on imaging purpose information, quality information based on imaging environment information, and quality information based on imaging method information. Moreover, the controller 211 may also use an internal ID like the purpose ID to manage in advance a combination of types of quality information to be acquired, and may issue an ID for internal management of the acquired quality information itself.

According to the present modification as above, the controller 211 automatically determines and acquires the quality information according to the imaging purpose from the imaging order. For this reason, the structure of the imaging order can be simplified, while quality information corresponding to the imaging purpose can be acquired.

In the above description, an example is disclosed in which the storage 212 (semiconductor memory, HDD) is used as the computer-readable medium of a program according to the present invention, but the computer-readable medium is not limited thereto. A portable recording medium such as a CD-ROM can be applied as another type of computer-readable medium. Furthermore, a carrier wave is also applied to the present invention as a medium for providing the data of a program according to the present invention through a communication channel.

Note that the descriptions in the above embodiment and modification are non-limiting examples of a favorable radiographic imaging apparatus, decision support method, and recording medium according to the present invention. For example, the configurations of the above embodiment and modification may also be combined, as appropriate.

Also, the above embodiment and modification describe a configuration in which the user moves the rounds car RC and adjusts the position, height, and direction of the tube 23 through the tube support 24, but are not limited thereto. In one configuration, the rounds car RC may also include a wheel driving mechanism that drives the wheels and a tube state driving mechanism that drives the state of the tube 23 and the tube support 24 (such as the position of the tube support 24, the horizontal position, height, and direction of the tube 23, and the aperture of the collimator 25). In this configuration, in step S31 of the navigation process, the controller 211 may be configured to move the rounds car RC two-dimensionally in the horizontal direction and change the state of the tube 23 and the tube support 24 to automatically set at least one of the placement location of the rounds car RC, the position of the tube support 24, the position and height of the tube 23, the aperture, and the SID through control of the wheel driving mechanism and the tube state driving mechanism according to the imaging support information rather than imaging operations by the user, and also control the generator 213 to automatically set the dose of the tube 23.

For example, in steps S30 and S31, the controller 211 controls the wheel driving mechanism, the tube state driving mechanism, and the generator 213 such that various information regarding the current radiographic imaging (such as the placement location of the rounds car RC, the position of the tube support 24, the position, height, and direction of the tube 23, the aperture, the SID, and the dose) is the same as the various information regarding the radiographic imaging of optical image data in the past for comparison in the imaging support information. According to this configuration, the user burden for radiographic imaging can be lessened and radiographic imaging of high image quality can be performed.

Additionally, in the configuration in which the rounds car RC is provided with the tube state driving mechanism as above, the controller 211 may be configured to change the radiation irradiation direction of the tube through control of the tube state driving mechanism, and forcibly lower the speed of angular change of the tube 23 when the angle of opposition between the FPD 1 and the tube 23 approaches a preset, prescribed angle based on acceleration information about the FPD 1 from the sensor 17 and acceleration information about the tube 23 from the sensor 27. With this configuration, for example, fine adjustments to the angle of opposition between the FPD 1 and the tube 23 can be made easily by operation input from the user through the input device 32, and radiographic imaging of high image quality can be performed.

Also, the above embodiment and modification describe a configuration in which the quality information includes past radiograph data and optical image data, but are not limited thereto. In one configuration, the quality information may include the image data of other examination information as the image data. The image data of other examination information is, for example, ultrasound image data from an ultrasound apparatus, three-dimensional image data obtained by computed tomography (CT) for identifying the location of a lesion (an embodiment of a radiographic imaging location).

Other details of the configuration and operation of each component forming the radiographic imaging system 100 in the above embodiment and modification can also be modified, as appropriate, without departing from the gist of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2022-019208, filed on Feb. 10, 2022, Japanese Patent Application No. 2022-019212, filed on Feb. 10, 2022, and Japanese Patent Application No. 2022-019216, filed on Feb. 10, 2022, including description, claims, drawings and abstract is incorporated herein by reference in its entirety.

1. A radiographic imaging apparatus comprising:
a hardware processor that acquires rounds imaging information related to radiographic imaging of an imaging subject when making rounds, and outputs, based on the acquired rounds imaging information, decision support information supporting a decision regarding whether radiograph data generated by the radiographic imaging satisfies a prescribed image quality.

2. The radiographic imaging apparatus according to claim 1, wherein the hardware processor determines a type of decision support information to be generated based on the rounds imaging information.

3. The radiographic imaging apparatus according to claim 1, wherein the hardware processor uses the rounds imaging information to generate the decision support information.

4. The radiographic imaging apparatus according to claim 3, wherein the hardware processor generates decision support information by selecting one from among using the rounds imaging information directly, processing the rounds imaging information, and combining and processing the radiograph data of the radiographic imaging and the rounds imaging information.

5. The radiographic imaging apparatus according to claim 1, wherein the rounds imaging information includes at least one of information for performing radiographic imaging, information associated with the radiograph data obtained as a result of performing the radiographic imaging, and information related to the radiographic imaging apparatus.

6. The radiographic imaging apparatus according to claim 1, wherein the decision support information is support information for making a decision regarding whether image retake due to imaging error in the radiograph data is necessary.

7. The radiographic imaging apparatus according to claim 1, wherein the hardware processor displays the decision support information on a display.

8. The radiographic imaging apparatus according to claim 7, wherein
the decision support information includes radiograph data of the imaging subject in the past or of another imaging subject, and
the hardware processor displays the generated radiograph data and the past radiograph data in association with each other.

9. The radiographic imaging apparatus according to claim 8, wherein the radiograph data of the imaging subject in the past or of another imaging subject includes radiograph data of normal or abnormal disease condition in the past.

10. The radiographic imaging apparatus according to claim 8, wherein the hardware processor superimposes an assistance image supporting the decision-making onto the radiograph data.

11. A decision support method comprising:
acquiring rounds imaging information related to radiographic imaging of an imaging subject when making rounds, and
outputting, based on the acquired rounds imaging information, decision support information supporting a decision regarding whether radiograph data generated by the radiographic imaging satisfies a prescribed image quality.

12. A computer readable recording medium storing a program causing a computer to function as a controller that,
acquires rounds imaging information related to radiographic imaging of an imaging subject when making rounds, and
outputs, based on the acquired rounds imaging information, decision support information supporting a decision regarding whether radiograph data generated by the radiographic imaging satisfies a prescribed image quality.
